SEQUENCE LISTING (33 sequences)

SEQ ID NO: 1 - protein, 23 aa, artificial
  Sp 1  LAL WT signal peptide
MKMRFLGLVVCLVLWTLHSEGSG

SEQ ID NO: 2 - protein, 24 aa, artificial
  Sp 2  alpha-1-antytripsin signal peptide
MPSSVSWGILLLAGLCCLVPVSLA

SEQ ID NO: 3 - protein, 25 aa, artificial
  Sp 6  iduronate 2-sulfatase signal peptide
MPPPRTGRGLLWLGLVLSSVCVALG

SEQ ID NO: 4 - protein, 18 aa, artificial
  Sp 7  chymotrypsinogen B2 signal peptide
MAFLWLLSCWALLGTTFG

SEQ ID NO: 5 - protein, 22 aa, artificial
  Sp 8  plasma protease inhibitor signal peptide
MASRLTLLTLLLLLLAGDRASS

SEQ ID NO: 6 - protein, 18 aa, artificial
  Sp 9  serum albumin preproprotein signal peptide
MKWVTFISLLFLFSSAYS

SEQ ID NO: 7 - protein, 19 aa, artificial
  Sp 10  azurocidin preproprotein signal peptide
MTRLTVLALLAGLLASSRA

SEQ ID NO: 8 - protein, 399 aa, Homo sapiens
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 9 - protein, 376 aa, artificial
  hLAL devoid of sp1
GKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 10 - DNA, 1200 bp, Homo sapiens
atgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtaa

SEQ ID NO: 11 - DNA, 75 bp, artificial
  Sp6 coding sequence
atgccgccaccccggaccggccgaggccttctctggctgggtctggttctgagctccgtctgcgtcgccctcgga

SEQ ID NO: 12 - DNA, 54 bp, artificial
  Sp7 coding sequence
atggctttcctctggctcctctcctgctgggccctcctgggtaccaccttcggc

SEQ ID NO: 13 - DNA, 66 bp, artificial
  sp8 coding sequence
atggcctccaggctgaccctgctgaccctcctgctgctgctgctggctggggatagagcctcctca

SEQ ID NO: 14 - DNA, 1131 bp, artificial
  WT hLAL w/o signal peptide
gggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtaa

SEQ ID NO: 15 - DNA, 1131 bp, artificial
  hLAL w/o signal peptide (from Opt_1)
gggaagctcacggcagtagacccggagaccaatatgaatgttagcgagatcattagttattggggtttcccaagtgaagagtaccttgtcgaaacagaagatggctacattttgtgcttgaataggattccacatggaagaaagaatcactccgataagggcccaaaaccggttgtatttctccagcacggcctgctggcagactctagtaattgggtgaccaacctcgccaatagttctttgggcttcattctggccgacgctggtttcgacgtatggatgggaaatagccggggaaatacgtggagccgaaagcataaaacgctgtccgttagccaagacgagttctgggcatttagctatgacgagatggctaaatatgatcttccagcctcaattaactttatcctgaataagacaggacaggagcaagtatactacgtcggccacagtcaagggacaactataggctttattgcattttcacagattcccgaattggctaaaaggatcaaaatgttctttgcattgggtcccgtggcatctgtagcgttttgtaccagcccgatggcgaagctggggcgcttgcccgatcatctgattaaagatttgtttggtgacaaggaatttctgcctcaatccgcgttccttaagtggctcgggactcatgtttgcacccatgtaatactgaaggaactgtgcgggaatctttgcttcttgttgtgcgggtttaatgaaaggaatcttaacatgagccgagtggatgtatacacgacacatagccccgctgggactagcgtgcaaaatatgctccattggtcccaagctgtgaaatttcaaaaatttcaggctttcgactggggctcttcagctaaaaattatttccactataaccagtcatatccacctacgtacaacgtaaaagacatgcttgttcccacggcagtctggagtggcggccacgattggttggcagacgtgtacgatgtcaatatcttgcttacccagattacaaacctcgtcttccatgagagtataccggaatgggagcatcttgatttcatttggggcttggacgcaccgtggcgattgtataataagatcataaaccttatgcggaaatatcagtaa

SEQ ID NO: 16 - DNA, 1131 bp, artificial
  hLAL w/o signal peptide (from Opt_2)
ggcaagctgaccgccgtggatcctgaaaccaacatgaacgtgtccgagatcatcagctactggggcttcccttctgaagagtacctggtggaaacagaggacggctacatcctgtgcctgaatagaatcccccacggcagaaagaaccactctgataagggccctaagcccgtggtctttctgcagcacggcctgctggccgacagcagcaactgggtgacaaacctcgctaattctagcctgggcttcatcctggccgatgccggcttcgacgtgtggatgggcaatagccgtggaaatacctggtcccggaagcacaagaccctgagcgtgagccaggacgagttctgggccttcagctatgatgagatggccaagtacgacctgcctgcttctatcaacttcattctgaacaagacaggccaggagcaggtgtactacgtgggccacagccagggcaccaccatcggctttatcgcctttagccagatccctgagctggccaagcggatcaagatgttcttcgccctgggacctgtggcctctgtggccttctgcacaagccctatggctaagctgggcagactgcccgaccacctgatcaaggacctgttcggcgacaaggaattcctgcctcagagcgccttcctgaagtggctgggcacacacgtgtgcacccatgtgatcctgaaagaactgtgtggcaacctgtgcttcctgctgtgcggctttaacgagagaaacctgaacatgagcagagtggacgtgtacaccacacatagccccgccggcaccagcgtgcagaacatgctgcactggtcccaggccgtgaagttccagaagttccaagcttttgactggggaagcagcgccaaaaactacttccactacaaccagagctacccccccacctacaatgtgaaagacatgctcgtgcctaccgccgtgtggagcggcggccacgactggctggccgacgtgtatgacgtgaacatcctgctgacacagataacaaacctggtgttccacgagagcatcccagaatgggagcacctggacttcatctggggcctggacgccccttggcggctgtacaacaagatcatcaacctgatgagaaaataccagtaa

SEQ ID NO: 17 - DNA, 1131 bp, artificial
  hLAL w/o signal peptide (from Opt_3)
ggcaagctgacagccgtggatcccgagacaaacatgaacgtgtccgagatcatcagctactggggcttccccagcgaggaatacctggtggaaaccgaggacggctacatcctgtgcctgaacagaatccctcacggccggaagaaccacagcgacaagggacctaagcctgtggtgtttctgcagcacggactgctggccgacagcagcaattgggtcaccaacctggccaatagcagcctgggcttcattctggccgatgccggcttcgatgtgtggatgggcaacagcagaggcaacacctggtccagaaagcacaagaccctgagcgtgtcccaggacgagttctgggccttcagctacgacgagatggccaaatacgatctgcccgccagcatcaacttcatcctgaacaagaccggccaagagcaggtctactacgtgggccactctcagggcaccaccatcggctttatcgcattctctcagatccccgagctggccaagcggatcaagatgttctttgctctgggccccgtggccagcgtggccttctgtacatctcctatggccaagctgggcagactgcccgaccacctgatcaaggatctgttcggcgacaaagagttcctgcctcagagcgccttcctgaagtggctgggaacccatgtgtgcacccacgtgatcctgaaagagctgtgcggcaacctgtgcttcctgctgtgtggcttcaacgagcggaacctgaacatgagcagagtggacgtgtacaccacacacagccctgccggaaccagcgtgcagaacatgctgcattggagccaggccgtgaagttccagaagtttcaggccttcgactggggcagcagcgccaagaactacttccactacaaccagagctacccgcctacctacaacgtgaaggacatgctggtgcccaccgccgtttggagcggaggacatgattggctggctgacgtgtacgacgtgaacatcctgctgacccagatcaccaatctggtgttccacgagagcatccccgagtgggagcacctggatttcatctggggactcgacgccccttggcggctgtacaacaagatcatcaacctgatgcggaagtaccagtaa

SEQ ID NO: 18 - DNA, 66 bp, artificial
  Sp8 (from Opt_1)
atggcatcacgcttgacgttgttgacactgcttctcttgcttctggcgggggacagggcctcaagc

SEQ ID NO: 19 - DNA, 66 bp, artificial
  Sp8 (from Opt_2)
atggccagcagactgaccctgctgaccctgctgctgctgctgcttgccggagatagagcctcttct

SEQ ID NO: 20 - DNA, 66 bp, artificial
  Sp8 (from Opt_3)
atggccagcagactgaccctgctgacactgcttctgctgctgctggcaggcgatagagcctcttct

SEQ ID NO: 21 - DNA, 1197 bp, artificial
  hLAL+Sp8 (from Opt_1)
atggcatcacgcttgacgttgttgacactgcttctcttgcttctggcgggggacagggcctcaagcgggaagctcacggcagtagacccggagaccaatatgaatgttagcgagatcattagttattggggtttcccaagtgaagagtaccttgtcgaaacagaagatggctacattttgtgcttgaataggattccacatggaagaaagaatcactccgataagggcccaaaaccggttgtatttctccagcacggcctgctggcagactctagtaattgggtgaccaacctcgccaatagttctttgggcttcattctggccgacgctggtttcgacgtatggatgggaaatagccggggaaatacgtggagccgaaagcataaaacgctgtccgttagccaagacgagttctgggcatttagctatgacgagatggctaaatatgatcttccagcctcaattaactttatcctgaataagacaggacaggagcaagtatactacgtcggccacagtcaagggacaactataggctttattgcattttcacagattcccgaattggctaaaaggatcaaaatgttctttgcattgggtcccgtggcatctgtagcgttttgtaccagcccgatggcgaagctggggcgcttgcccgatcatctgattaaagatttgtttggtgacaaggaatttctgcctcaatccgcgttccttaagtggctcgggactcatgtttgcacccatgtaatactgaaggaactgtgcgggaatctttgcttcttgttgtgcgggtttaatgaaaggaatcttaacatgagccgagtggatgtatacacgacacatagccccgctgggactagcgtgcaaaatatgctccattggtcccaagctgtgaaatttcaaaaatttcaggctttcgactggggctcttcagctaaaaattatttccactataaccagtcatatccacctacgtacaacgtaaaagacatgcttgttcccacggcagtctggagtggcggccacgattggttggcagacgtgtacgatgtcaatatcttgcttacccagattacaaacctcgtcttccatgagagtataccggaatgggagcatcttgatttcatttggggcttggacgcaccgtggcgattgtataataagatcataaaccttatgcggaaatatcagtaa

SEQ ID NO: 22 - DNA, 1197 bp, artificial
  hLAL+Sp8 (from Opt_2)
atggccagcagactgaccctgctgaccctgctgctgctgctgcttgccggagatagagcctcttctggcaagctgaccgccgtggatcctgaaaccaacatgaacgtgtccgagatcatcagctactggggcttcccttctgaagagtacctggtggaaacagaggacggctacatcctgtgcctgaatagaatcccccacggcagaaagaaccactctgataagggccctaagcccgtggtctttctgcagcacggcctgctggccgacagcagcaactgggtgacaaacctcgctaattctagcctgggcttcatcctggccgatgccggcttcgacgtgtggatgggcaatagccgtggaaatacctggtcccggaagcacaagaccctgagcgtgagccaggacgagttctgggccttcagctatgatgagatggccaagtacgacctgcctgcttctatcaacttcattctgaacaagacaggccaggagcaggtgtactacgtgggccacagccagggcaccaccatcggctttatcgcctttagccagatccctgagctggccaagcggatcaagatgttcttcgccctgggacctgtggcctctgtggccttctgcacaagccctatggctaagctgggcagactgcccgaccacctgatcaaggacctgttcggcgacaaggaattcctgcctcagagcgccttcctgaagtggctgggcacacacgtgtgcacccatgtgatcctgaaagaactgtgtggcaacctgtgcttcctgctgtgcggctttaacgagagaaacctgaacatgagcagagtggacgtgtacaccacacatagccccgccggcaccagcgtgcagaacatgctgcactggtcccaggccgtgaagttccagaagttccaagcttttgactggggaagcagcgccaaaaactacttccactacaaccagagctacccccccacctacaatgtgaaagacatgctcgtgcctaccgccgtgtggagcggcggccacgactggctggccgacgtgtatgacgtgaacatcctgctgacacagataacaaacctggtgttccacgagagcatcccagaatgggagcacctggacttcatctggggcctggacgccccttggcggctgtacaacaagatcatcaacctgatgagaaaataccagtaa

SEQ ID NO: 23 - DNA, 1197 bp, artificial
  hLAL+Sp8 (from Opt_3)
atggccagcagactgaccctgctgacactgcttctgctgctgctggcaggcgatagagcctcttctggcaagctgacagccgtggatcccgagacaaacatgaacgtgtccgagatcatcagctactggggcttccccagcgaggaatacctggtggaaaccgaggacggctacatcctgtgcctgaacagaatccctcacggccggaagaaccacagcgacaagggacctaagcctgtggtgtttctgcagcacggactgctggccgacagcagcaattgggtcaccaacctggccaatagcagcctgggcttcattctggccgatgccggcttcgatgtgtggatgggcaacagcagaggcaacacctggtccagaaagcacaagaccctgagcgtgtcccaggacgagttctgggccttcagctacgacgagatggccaaatacgatctgcccgccagcatcaacttcatcctgaacaagaccggccaagagcaggtctactacgtgggccactctcagggcaccaccatcggctttatcgcattctctcagatccccgagctggccaagcggatcaagatgttctttgctctgggccccgtggccagcgtggccttctgtacatctcctatggccaagctgggcagactgcccgaccacctgatcaaggatctgttcggcgacaaagagttcctgcctcagagcgccttcctgaagtggctgggaacccatgtgtgcacccacgtgatcctgaaagagctgtgcggcaacctgtgcttcctgctgtgtggcttcaacgagcggaacctgaacatgagcagagtggacgtgtacaccacacacagccctgccggaaccagcgtgcagaacatgctgcattggagccaggccgtgaagttccagaagtttcaggccttcgactggggcagcagcgccaagaactacttccactacaaccagagctacccgcctacctacaacgtgaaggacatgctggtgcccaccgccgtttggagcggaggacatgattggctggctgacgtgtacgacgtgaacatcctgctgacccagatcaccaatctggtgttccacgagagcatccccgagtgggagcacctggatttcatctggggactcgacgccccttggcggctgtacaacaagatcatcaacctgatgcggaagtaccagtaa

SEQ ID NO: 24 - protein, 20 aa, artificial
  hLAL (sequence mutated in isoform 2)
DGYILCLNRIPHGRKNHSDK

SEQ ID NO: 25 - protein, 20 aa, artificial
  hLAL variant isoform 2
MACLEFVPFDVQMCLEFLPS

SEQ ID NO: 26 - protein, 343 aa, Homo sapiens
MACLEFVPFDVQMCLEFLPSGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 27 - DNA, 1318 bp, artificial
  Opti-1 (KOZAK + optimized sequence + HA tag).
caccatggcatcacgcttgacgttgttgacactgcttctcttgcttctggcgggggacagggcctcaagcgggaagctcacggcagtagacccggagaccaatatgaatgttagcgagatcattagttattggggtttcccaagtgaagagtaccttgtcgaaacagaagatggctacattttgtgcttgaataggattccacatggaagaaagaatcactccgataagggcccaaaaccggttgtatttctccagcacggcctgctggcagactctagtaattgggtgaccaacctcgccaatagttctttgggcttcattctggccgacgctggtttcgacgtatggatgggaaatagccggggaaatacgtggagccgaaagcataaaacgctgtccgttagccaagacgagttctgggcatttagctatgacgagatggctaaatatgatcttccagcctcaattaactttatcctgaataagacaggacaggagcaagtatactacgtcggccacagtcaagggacaactataggctttattgcattttcacagattcccgaattggctaaaaggatcaaaatgttctttgcattgggtcccgtggcatctgtagcgttttgtaccagcccgatggcgaagctggggcgcttgcccgatcatctgattaaagatttgtttggtgacaaggaatttctgcctcaatccgcgttccttaagtggctcgggactcatgtttgcacccatgtaatactgaaggaactgtgcgggaatctttgcttcttgttgtgcgggtttaatgaaaggaatcttaacatgagccgagtggatgtatacacgacacatagccccgctgggactagcgtgcaaaatatgctccattggtcccaagctgtgaaatttcaaaaatttcaggctttcgactggggctcttcagctaaaaattatttccactataaccagtcatatccacctacgtacaacgtaaaagacatgcttgttcccacggcagtctggagtggcggccacgattggttggcagacgtgtacgatgtcaatatcttgcttacccagattacaaacctcgtcttccatgagagtataccggaatgggagcatcttgatttcatttggggcttggacgcaccgtggcgattgtataataagatcataaaccttatgcggaaatatcagggagggagcggctatccctatgacgtgcctgattacgccggcacaggatcctacccctatgatgtgcctgactacgctggcagcgccggatacccttatgatgtgcctgattatgcttaa

SEQ ID NO: 28 - DNA, 1318 bp, artificial
  Opti-2 (KOZAK + optimized sequence + HA tag).
caccatggccagcagactgaccctgctgaccctgctgctgctgctgcttgccggagatagagcctcttctggcaagctgaccgccgtggatcctgaaaccaacatgaacgtgtccgagatcatcagctactggggcttcccttctgaagagtacctggtggaaacagaggacggctacatcctgtgcctgaatagaatcccccacggcagaaagaaccactctgataagggccctaagcccgtggtctttctgcagcacggcctgctggccgacagcagcaactgggtgacaaacctcgctaattctagcctgggcttcatcctggccgatgccggcttcgacgtgtggatgggcaatagccgtggaaatacctggtcccggaagcacaagaccctgagcgtgagccaggacgagttctgggccttcagctatgatgagatggccaagtacgacctgcctgcttctatcaacttcattctgaacaagacaggccaggagcaggtgtactacgtgggccacagccagggcaccaccatcggctttatcgcctttagccagatccctgagctggccaagcggatcaagatgttcttcgccctgggacctgtggcctctgtggccttctgcacaagccctatggctaagctgggcagactgcccgaccacctgatcaaggacctgttcggcgacaaggaattcctgcctcagagcgccttcctgaagtggctgggcacacacgtgtgcacccatgtgatcctgaaagaactgtgtggcaacctgtgcttcctgctgtgcggctttaacgagagaaacctgaacatgagcagagtggacgtgtacaccacacatagccccgccggcaccagcgtgcagaacatgctgcactggtcccaggccgtgaagttccagaagttccaagcttttgactggggaagcagcgccaaaaactacttccactacaaccagagctacccccccacctacaatgtgaaagacatgctcgtgcctaccgccgtgtggagcggcggccacgactggctggccgacgtgtatgacgtgaacatcctgctgacacagataacaaacctggtgttccacgagagcatcccagaatgggagcacctggacttcatctggggcctggacgccccttggcggctgtacaacaagatcatcaacctgatgagaaaataccagggagggagcggctatccctatgacgtgcctgattacgccggcacaggatcctacccctatgatgtgcctgactacgctggcagcgccggatacccttatgatgtgcctgattatgcttaa

SEQ ID NO: 29 - DNA, 1318 bp, artificial
  Opti3 (KOZAK + optimized sequence + HA tag)
caccatggccagcagactgaccctgctgacactgcttctgctgctgctggcaggcgatagagcctcttctggcaagctgacagccgtggatcccgagacaaacatgaacgtgtccgagatcatcagctactggggcttccccagcgaggaatacctggtggaaaccgaggacggctacatcctgtgcctgaacagaatccctcacggccggaagaaccacagcgacaagggacctaagcctgtggtgtttctgcagcacggactgctggccgacagcagcaattgggtcaccaacctggccaatagcagcctgggcttcattctggccgatgccggcttcgatgtgtggatgggcaacagcagaggcaacacctggtccagaaagcacaagaccctgagcgtgtcccaggacgagttctgggccttcagctacgacgagatggccaaatacgatctgcccgccagcatcaacttcatcctgaacaagaccggccaagagcaggtctactacgtgggccactctcagggcaccaccatcggctttatcgcattctctcagatccccgagctggccaagcggatcaagatgttctttgctctgggccccgtggccagcgtggccttctgtacatctcctatggccaagctgggcagactgcccgaccacctgatcaaggatctgttcggcgacaaagagttcctgcctcagagcgccttcctgaagtggctgggaacccatgtgtgcacccacgtgatcctgaaagagctgtgcggcaacctgtgcttcctgctgtgtggcttcaacgagcggaacctgaacatgagcagagtggacgtgtacaccacacacagccctgccggaaccagcgtgcagaacatgctgcattggagccaggccgtgaagttccagaagtttcaggccttcgactggggcagcagcgccaagaactacttccactacaaccagagctacccgcctacctacaacgtgaaggacatgctggtgcccaccgccgtttggagcggaggacatgattggctggctgacgtgtacgacgtgaacatcctgctgacccagatcaccaatctggtgttccacgagagcatccccgagtgggagcacctggatttcatctggggactcgacgccccttggcggctgtacaacaagatcatcaacctgatgcggaagtaccagggagggagcggctatccctatgacgtgcctgattacgccggcacaggatcctacccctatgatgtgcctgactacgctggcagcgccggatacccttatgatgtgcctgattatgcttaa

SEQ ID NO: 30 - protein, 401 aa, artificial
  SP6+hLAL protein
MPPPRTGRGLLWLGLVLSSVCVALGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 31 - protein, 394 aa, artificial
  SP7+hLAL protein
MAFLWLLSCWALLGTTFGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 32 - protein, 398 aa, artificial
  SP8+hLAL protein
MASRLTLLTLLLLLLAGDRASSGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 33 - DNA, 1197 bp, artificial
  coding sequence sp8+LAL
atggcctccaggctgaccctgctgaccctcctgctgctgctgctggctggggatagagcctcctcagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtaa

(19) United States
(12) Patent Application Publication
(10) Pub. No.: US 2024/0067942 A1
(43) Pub. Date: Feb. 29, 2024

(54) LYSOSOMAL ACID LIPASE VARIANTS AND USES THEREOF

(71) Applicants: GENETHON, EVRY-COURCOURONNES (FR); INSERM (INSTITUT NATIONAL DE LA SANTÉ ET DE LA RECHERCHE MÉDICALE), PARIS (FR); UNIVERSITE D'EVRY VAL D'ESSONNE, EVRY-COURCOURONNES (FR); UNIVERSITE PARIS-SACLAY, GIF SUR YVETTE (FR)

(72) Inventors: MARIO AMENDOLA, PARIS (FR); MARINE LAURENT, VIRY-CHATILLON (FR); GIULIA PAVANI, LANSDOWNE, PA (US)

(21) Appl. No.: 18/266,282

(22) PCT Filed: Dec. 9, 2021
(86) PCT No.: PCT/EP2021/084929

§ 371 Date: Jun. 9, 2023

(30) Foreign Application Priority Data
Dec. 9, 2020 (EP) 20306524.8

(51) Int. Cl. C12N 9/20; A61P 3/00; C12N 5/0789; C12N 15/86

(52) U.S. Cl. CPC C12N 9/20; A61P 3/00; C12N 5/0647; C12N 15/86; C12N 2710/10041; C12Y 301/01013

(57) ABSTRACT

The present invention relates to variants of lysosomal acid lipase (LAL) and uses thereof.

The present invention relates to nucleic acid and protein variants of lysosomal acid lipase (LAL) and uses thereof. Said variants are linked to heterologous signal peptides.

### BACKGROUND OF THE INVENTION

Lysosomal acid lipase (LAL) hydrolyzes cholesterol esters (CE) and triglycerides (TG) that are internalized into the lysosome via low-density lipoprotein (LDL) receptors. LAL enzyme requires post-translational modifications (N-glycosylation) to be enzymatically active, then traffics through the lysosome or is secreted out of cells.

LAL plays a critical role in lipid homeostasis. Therefore, mutations in LIPA gene (OMIM ID: 613497, GenBank Accession no. NG_008194) encoding for the LAL enzyme causes severe metabolic disorders. LAL deficiency (LAL-D) is an autosomal recessive lysosomal storage disorder resulting in missing or deficient LAL enzyme. The phenotypic spectrum of LAL-D ranges from the severe infantile-onset form (known as Wolman Disease, WD) to later-onset forms (referred to as cholesterol ester storage disease, CESD). Wolman Disease (≤5% LAL activity), the most severe form affecting ˜1/300,000 live births, is characterized by malnutrition, hepatomegaly, liver disease and cortical insufficiency. Without treatment, Wolman Disease is fatal within the first year of life. Cholesteryl ester storage disease, a late onset LAL deficiency, can be identified in various stages of life with varying degrees of severity. The morbidity of late-onset CESD results from atherosclerosis (coronary artery disease, stroke), liver disease (e.g., altered liver function, steatosis, fibrosis, cirrhosis and related complications of oesophageal varices, and/or liver failure), complications of secondary hypersplenism (i.e., anaemia and/or thrombocytopenia), and/or malabsorption.

Donor-derived hematopoietic stem cell transplantation (HSCT) and liver transplantation have been used in a small number of cases, but outcomes remain very poor, with few reported survivors of HSCT (Krivit et al. Bone Marrow Transplantation. 1992;10 Suppl 1:97-101; Gramatges et al. Bone Marrow Transplant. 2009;44(7):449-450; Yanir et al. Mol Genet Metab. 2013;109(2):224-226). Most of the deaths resulted from either progression of LAL deficiency or HSCT-related complications such as infection, graft-versus-host disease, and/or graft failure. In addition, transplantation treatment is severely limited by donor availability.

In December 2015, enzyme replacement therapy with recombinant LAL (sebelipase alpha) has been approved by the Food and Drug Administration for management of LAL deficiency (Jones et al. Orphanet J Rare Dis. 2017; 12:25). This treatment consists in intravenous injection of the missing LAL enzyme that is uptaken by affected cells and eliminates accumulated toxic substrates (cross-correction). Enzyme replacement therapy is the only symptomatic treatment available. However, the benefit of enzyme replacement therapy is limited by the need for frequent infusions since it requires a weekly injection of the recombinant human LAL enzyme throughout the patient's life. Its long-term efficacy is still being evaluated, but initial evidence shows that efficacy decreases over time in some patients, probably due to an anti-drug immune response.

With the aim to provide a long-term treatment of LAL deficiency which improves the quality of life of patients, the present inventors provided another treatment approach based on gene therapy. For example, the present inventors provided an ex vivo gene therapy aiming at introducing a functional copy of the LIPA gene into patients' hematopoietic stem cells (HSCs) (Pavani et al.; Nat Commun. 2020;11(1):3778). Autologous HSCs can be easily accessed for ex vivo gene manipulation and re-administration, thus circumventing immunological issues. The corrected HSCs are intended to be administered into the bloodstream, reach and multiply in the bone marrow to produce new corrected cells in the blood. The LAL enzyme will then be secreted into the bloodstream in order to be recovered by LAL-deficient cells and ultimately restore metabolic function.

However, LAL enzyme is mostly intracellular, and only a small fraction is secreted in the blood and available for cross-correction. There is thus a need in providing a long term treatment of LAL deficiency, with high expression and secretion levels of the therapeutic LAL enzyme. In particular, there is a need to improve the expression and secretion of LAL without affecting its enzymatic activity, nor its ability to cross-correct LAL deficient cells.

### SUMMARY OF THE INVENTION

A first aspect of the invention relates to a nucleic acid molecule encoding a functional chimeric LAL protein, comprising a signal peptide moiety and a functional LAL moiety, wherein the signal peptide moiety has an amino acid sequence selected in the group consisting of SEQ ID NO:3 to 5, or wherein the signal peptide moiety is a functional signal peptide moiety having an amino acid sequence comprising from 1 to 5, in particular from 1 to 4, in particular from 1 to 3, in particular from 1 to 2, in particular 1 amino acid deletion(s), insertion(s) or substitution(s) as compared to the sequence of SEQ ID NO:3 to 5. In a preferred embodiment, the signal peptide moiety has the amino acid sequence of SEQ ID NO:5, or is a functional signal peptide moiety having an amino acid sequence comprising from 1 to 5, in particular from 1 to 4, in particular from 1 to 3, in particular from 1 to 2, in particular 1 amino acid deletion(s), insertion(s) or substitution(s) as compared to the sequence of SEQ ID NO:5.

In a particular embodiment, the functional LAL moiety is a functional human LAL moiety, preferably a functional human LAL moiety devoid of its natural signal peptide. In a particular embodiment, the functional LAL moiety comprises or consists of SEQ ID NO:9, or comprises or consists of an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:9.

In a particular embodiment, the nucleic acid molecule of the invention comprises a nucleotide sequence resulting from the combination of:
a signal peptide moiety coding sequence selected in the group consisting of SEQ ID NO:11 to 13 and SEQ ID NO:18 to 20; and
a LAL moiety coding sequence selected in the group consisting of SEQ ID NO:14 to 17.

In a particular embodiment, the nucleic acid molecule is a nucleotide sequence optimized to improve the expression of the functional chimeric LAL protein in vivo, in particular the nucleotide sequence selected in the group consisting of SEQ ID NO:21 to 23, preferably SEQ ID NO:22 or SEQ ID NO:23.

The invention also relates to a nucleic acid construct comprising the nucleic acid molecule of the invention, which is in particular an expression cassette comprising said nucleic acid molecule operably linked to a promoter, such as an ubiquitous promoter, a liver-specific promoter or an erythroid-specific promoter, wherein said nucleic acid construct optionally further comprises an intron and/or post-transcriptional regulatory sequence(s).

The invention also relates to a vector comprising the nucleic acid molecule or the nucleic acid construct as described herein, which is preferably a viral vector, preferably a retroviral vector, such as a lentiviral vector, or an AAV vector, such as a single-stranded or double-stranded self-complementary AAV vector, preferably an AAV vector with an AAV-derived capsid, such as an AAV6, AAV8, AAV9, AAV2 or AAV-DJ derived capsid.

Another aspect of the invention relates to a cell comprising the nucleic acid molecule, the nucleic acid construct or the vector as described herein, wherein the cell is in particular an hematopoietic stem cell (HSC). In a particular embodiment, the cell is a genetically modified hematopoietic stem cell comprising the nucleic acid molecule in at least one globin locus , the nucleic acid molecule being placed under the control of the endogenous promoter of the globin gene.

The invention also relates to a functional chimeric LAL protein, comprising a signal peptide moiety and a functional LAL moiety, wherein the signal peptide moiety has an amino acid sequence selected in the group consisting of SEQ ID NO:3 to 5, or wherein the signal peptide moiety is a functional signal peptide moiety having an amino acid sequence comprising from 1 to 5, in particular from 1 to 4, in particular from 1 to 3, in particular from 1 to 2, in particular 1 amino acid deletion(s), insertion(s) or substitution(s) as compared to the sequence of SEQ ID NO:3 to 5. In a particular embodiment, the functional LAL moiety is a functional human LAL moiety, preferably a functional human LAL moiety devoid of its natural signal peptide, more preferably a functional LAL moiety comprising or consisting of SEQ ID NO:9, or comprising or consisting of an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:9.

In a particular embodiment, the functional chimeric LAL protein comprises an amino acid sequence resulting from the combination of:
a signal peptide moiety sequence selected in the group consisting of SEQ ID NO:3 to 5; and
a LAL moiety sequence of SEQ ID NO:9.

The invention also relates to a pharmaceutical composition, comprising, in a pharmaceutically acceptable carrier, the nucleic acid molecule, the nucleic acid construct, the vector, the cell, or the chimeric polypeptide as described herein.

The invention also relates to the nucleic acid molecule, the nucleic acid construct, the vector, the cell, or the chimeric polypeptide as described herein for use as a medicament.

The invention also relates to the nucleic acid molecule, the nucleic acid construct, the vector, the cell, or the chimeric polypeptide as described herein, for use in a method for treating LAL-deficiency, such as for treating Wolman Disease (WD) or Cholesteryl Ester Storage Disease (CESD).

### LEGENDS TO THE FIGURES

FIG. 1. LAL Expression and activity in K562 cells at day 6 after lentiviral transduction. (A) Western blot of intracellular LAL (UT: untreated cells). (B) Quantification of secreted LAL. (C) Quantification of LAL activity in supernatant. Bars represents mean±SD

FIG. 2. LAL expression and activity in CD34+ cells at day 14 after lentiviral transduction. Western blot of LAL in supernatant (A) and cell lysate (B). (C) Quantification of LAL expression and secretion (A and B). Ratio over untreated cells. (D) Quantification of LAL activity in supernatant. Ratio over untreated cells. Bars represents mean±SD

FIG. 3. Cross-correction of patient cells. Mean intensity (MFI) of Nile red staining of Wolman patient's fibroblasts co-cultured with K562 cells transduced with lentiviral vector encoding for different chimeric LAL enzymes. Each dot represents a cells (UT n=88; Sp 1n=133; Sp 7 n=125; Sp8 n=130; healthy n=104). Bars represents mean±SD (***, p<0.001 NS: Non-significant, p>0.9999 ANOVA) UT: co-culturing with untransduced K562; Healthy: healthy fibroblasts.

FIG. 4. Expression and activity of codon optimized LIPA cDNA. LAL expression, secretion (A, B) and activity (C) in K562 transduced with lentiviral vectors encoding for different optimized LIPA cDNA sequences (days 11).

### DETAILED DESCRIPTION OF THE INVENTION

### Nucleic Acid Molecule

The present invention relates to a nucleic acid molecule encoding a chimeric LAL polypeptide. This chimeric LAL polypeptide comprises a functional LAL moiety fused to an heterologous signal peptide moiety. The inventors have surprisingly shown that fusion of a LAL sequence to an heterologous signal peptide, such as those described below, greatly improves LAL secretion and expression while preserving its enzymatic activity and its ability to cross-correct LAL deficient cells.

Lysosomal acid lipase (or “LAL”) also called “cholesteryl ester hydrolase” (EC 3.1.1.13) is an essential lysosomal enzyme that hydrolyzes cholesteryl esters and triglycerides that are internalized into the lysosome via low density lipoprotein (LDL) particles receptor-mediated endocytosis. In particular, LAL catalyses the deacylation of triacylglyceryl and cholesteryl ester core lipids of endocytosed low density lipoproteins to generate free fatty acids and cholesterol.

LAL is encoded by the gene known as “LIPA” (NCBI Accession no. U08464.1) which contains nine coding exons localized in chromosome 10. Mutations in this gene can result in a condition known as LAL deficiency characterized by the massive accumulation of cholesteryl esters and triglycerides in vital tissues of affected individuals including liver, spleen, gut, blood vessel walls, and other important organs. As a result, LAL deficiency is typically associated with significant morbidity and mortality, and can affect individuals from infancy through adulthood.

Extremely low levels of the LAL enzyme typically causes early onset of LAL Deficiency, sometimes called Wolman Disease (also known as Wolman's disease or Wolman's syndrome). Early onset LAL deficiency typically affects infants in the first year of life. For example, the build-up of fatty material in the cells of the gut prevents the body from absorbing nutrients. Consequently, Wolman disease is a rapidly progressive and typically fatal condition characterized by malabsorption, growth failure, and significant weight loss. These infants typically die during their first year of life from a failure to grow and from other complications due to liver failure.

Later onset LAL Deficiency is sometimes called Cholesteryl Ester Storage Disease (CESD) and can affect children and adults. Typically, CESD patients experience enlarged liver (hepatomegaly), cirrhosis, chronic liver failure, severe premature atherosclerosis, hardening of the arteries, or elevated levels of serum Low Density Lipoprotein (LDL). Children may also have calcium deposits in the adrenal glands and develop jaundice.

Allelic variations of human LIPA have been characterized and missense as well as nonsense and deletion mutations linked to WD and CESD have been identified (see, e.g., Lugowska et al., Lysosomal Storage Diseases (2012) Vol. 10: 1-8).

Typically, human LAL is first synthesized as a precursor protein of 399 amino acid residues (SEQ ID NO:8), containing a signal peptide at the N-terminus. The signal peptide is cleaved post-translationally resulting in the mature form of human LAL.

As mentioned above, the nucleic acid molecule of the invention encodes a chimeric LAL polypeptide, which comprises an heterologous signal peptide moiety fused to a functional LAL moiety.

The expression “functional LAL moiety” refers to any LAL polypeptide that has the functionality of wild-type LAL protein, in particular of human LAL (hLAL). As defined above, the functionality of wild-type LAL is to hydrolyse cholesteryl esters and triglyceride, to liberate fatty acids and cholesterol. The functional LAL moiety encoded by the nucleic acid of the invention may have a hydrolysing activity of at least 50%, 60%, 70%, 80%, 90%, 95%, 99%, or at least 100% as compared to the wild-type human LAL polypeptide. The activity of the LAL protein encoded by the nucleic acid of the invention may even be of more than 100%, such as of more than 110%, 120%, 130%, 140%, or even more than 150% of the activity of the wild-type human LAL polypeptide.

A skilled person is readily able to determine whether a nucleic acid molecule according to the invention expresses a functional LAL protein. Suitable methods would be apparent to those skilled in the art. For example, one suitable in vitro method involves inserting the nucleic acid into a vector, such as a plasmid or viral vector, transfecting or transducing host cells and assaying for LAL activity. LAL enzymatic activity can be determined by using a fluorogenic substrate such as the 4-methylumbelliferyl-oleate or the 4-methylumbelliferyl phosphate. Suitable methods are described in more details in the experimental part below.

The coding sequence of the functional LAL moiety can be derived from any source, including avian and mammalian species. The term “avian” as used herein includes, but is not limited to, chickens, ducks, geese, quail, turkeys and pheasants. The term “mammal” as used herein includes, but is not limited to, humans, simians and other non-human primates, bovines, ovines, caprines, equines, felines, canines, lagomorphs, etc. In particular embodiments of the invention, the nucleic acid molecule of the invention encodes a human, mouse or quail, in particular a human, LAL polypeptide. In a particular embodiment, the “functional LAL moiety” is a human functional LAL polypeptide.

The term “functional LAL moiety”, as used herein, encompasses mature and precursor LAL, as well as modified or mutated by insertion(s), deletion (s) and/or substitution(s) LAL proteins or fragments thereof that are functional derivatives of LAL, i.e. that retain biological function of LAL (i.e., cleaves the fatty acids from cholesteryl esters and triglycerides as defined above). Natural functional variants of human LAL polypeptide are known. For example, in some embodiments, residues 1-56 of SEQ ID NO:8 are deleted and/or residues 57-76 of SEQ ID NO:8 (DGYILCLNRIPHGRKNHSDK, SEQ ID NO:24) are replaced with MACLEFVPFDVQMCLEFLPS (SEQ ID NO:25). In particular, the natural functional variant may be the sequence of SEQ ID NO:26, corresponding to isoform 2 of LAL protein (Uniprot identifier: P38571-2). In some embodiments, residue 16 of SEQ ID NO:8 has a Thr to Pro substitution (Uniprot identifier: VAR_004247). In some embodiments, residue 23 of SEQ ID NO:8 has a Gly to Arg substitution (Uniprot identifier: VAR_026523). In some embodiments, residue 29 of SEQ ID NO:8, has a Val to Leu substitution (Uniprot identifier: VAR_026524). In some embodiments, residue 228 of SEQ ID NO:8, has a Phe to Ser substitution (Uniprot identifier: VAR_049821).

Furthermore, the chimeric LAL polypeptide encoded by the nucleic acid molecule as herein described may comprise a LAL moiety that is a functional, truncated form of LAL.

In a particular embodiment, a “precursor form of LAL” is a LAL polypeptide that comprises its natural signal peptide. For example, the sequence of SEQ ID NO:8 (399 amino acid residues long) is the precursor form of human LAL (hLAL).

In a particular embodiment of the invention, the functional LAL moiety encoded by the nucleic acid molecule of the invention corresponds to a mature form of LAL, in particular of hLAL. According to this embodiment, the functional LAL moiety corresponds to the precursor form of LAL polypeptide as defined above, but devoid of its natural signal peptide. In a particular embodiment, the functional LAL moiety is a functional human LAL moiety devoid of its natural signal peptide. The signal peptide of human LAL is any of the first 21 to 27 amino acid residues at the N-terminus of precursor hLAL of SEQ ID NO:8. After cleavage of the signal peptide, a mature form of hLAL can be 372 to 378 amino acid residues long, depending on the length of the signal peptide sequence cleaved off from the 399 amino acid residues long hLAL. Thus, the mature form of hLAL without the signal peptide can include hLAL having 378 amino acid residues (i.e., from Ser22 to Gln399 of SEQ ID NO:8), 377 amino acid residues (i.e., from Gly23 to Gln399 of SEQ ID NO:8), 376 amino acid residues (i.e., from Gly24 to Gln399 of SEQ ID NO:8), 375 amino acid residues (i.e., from Lys25 to Gln399 of SEQ ID NO:8), 374 amino acid residues (i.e., from Leu26 to Gln399 of SEQ ID NO:8), 373 amino acid residues (i.e., from Thr27 to Gln399 of SEQ ID NO:8), or 372 amino acid residues (i.e., from Ala28 to Gln399 of SEQ ID NO:8).

In a particular embodiment, the signal peptide of hLAL corresponds to the first 23 amino acid residues at the N-terminus of the precursor hLAL of SEQ ID NO:8. According to this embodiment, the signal peptide of hLAL is as shown in SEQ ID NO:1. Thus, in a particular embodiment, the functional LAL moiety is a functional human LAL moiety devoid of the natural signal peptide shown in SEQ ID NO:1.

According to a particular embodiment, the mature form of hLAL (without the signal peptide) corresponds to a sequence of 376 amino acid residues as referred in SEQ ID NO:9.

In a particular embodiment, the nucleic acid molecule encodes a functional LAL moiety comprising or consisting of SEQ ID NO:9, or comprising or consisting of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:9.

In a particular embodiment, the functional LAL moiety comprises or consists of SEQ ID NO:9, or a functional variant thereof comprising from 1 to 50, in particular from 1 to 340, in particular from 1 to 30, in particular from 1 to 20, in particular from 1 to 10 or from 1 to 5 amino acid substitutions as compared to the sequence shown in SEQ ID NO:9, such as 1, 2, 3, 4 or 5 amino acid substitutions.

The term “identical” and declinations thereof when referring to a polypeptide means that when a position in two compared polypeptide sequences is occupied by the same amino acid (e.g. if a position in each of two polypeptides is occupied by a leucine), then the polypeptides are identical at that position. The percent of identity between two polypeptides is a function of the number of matching positions shared by the two sequences divided by the number of positions compared ×100. For example, if 6 of 10 of the positions in two polypeptides are matched then the two sequences are 60% identical. Generally, a comparison is made when two sequences are aligned to give maximum identity. Various bioinformatic tools known to the one skilled in the art might be used to align nucleic acid sequences such as BLAST or FASTA.

In a particular embodiment of the invention, the nucleic acid molecule of the invention comprises a functional LAL moiety coding sequence, wherein the functional LAL moiety coding sequence has at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to nucleotides 70-1197 of the sequence shown in SEQ ID NO:10, which is the sequence coding the wild-type hLAL of SEQ ID NO:8 (nucleotides 1-69 of SEQ ID NO:10 being the part encoding for the natural signal peptide of hLAL). In a particular embodiment of the invention, the nucleic acid molecule of the invention comprises a functional LAL moiety coding sequence, wherein the functional LAL moiety coding sequence has at least 70, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to sequence of SEQ ID NO:14.

The term “nucleic acid sequence” (or nucleic acid molecule) refers to a DNA or RNA molecule in single or double stranded form, particularly a DNA encoding a chimeric LAL protein according to the invention.

The term “identical” and declinations thereof refers to the sequence identity between two nucleic acid molecules. When a position in both of the two compared sequences is occupied by the same base e.g., if a position in each of two DNA molecules is occupied by adenine, then the molecules are identical at that position. The percent of identity between two sequences is a function of the number of matching positions shared by the two sequences divided by the number of positions compared ×100. For example, if 6 of 10 of the positions in two sequences are matched then the two sequences are 60% identical. Generally, a comparison is made when two sequences are aligned to give maximum identity. Various bioinformatic tools known to the one skilled in the art might be used to align nucleic acid sequences such as BLAST or FASTA.

The sequence of the nucleic acid molecule of the invention, encoding a functional LAL moiety, may be optimized for expression of the LAL polypeptide in vivo. Sequence optimization may include a number of changes in a nucleic acid sequence, including codon optimization, increase of GC content, decrease of the number of CpG islands, decrease of the number of alternative open reading frames (ARFs) and decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimised sequence. In a preferred embodiment of the invention, such optimized nucleotide sequence encoding a functional LAL moiety is codon-optimized to improve its expression in human cells compared to non-codon optimized nucleotide sequences coding for the same functional LAL moiety, for example by taking advantage of the human specific codon usage bias.

In a particular embodiment, the optimized LAL coding sequence is codon optimized, and/or has an increased GC content and/or has a decreased number of alternative open reading frames, and/or has a decreased number of splice donor and/or splice acceptor sites, as compared to nucleotides 70-1197 of the wild-type hLAL coding sequence of SEQ ID NO:10. In addition to the GC content and/or number of ARFs, sequence optimization may also comprise a decrease in the number of CpG islands in the sequence and/or a decrease in the number of splice donor and acceptor sites. Of course, as is well known to those skilled in the art, sequence optimization is a balance between all these parameters, meaning that a sequence may be considered optimized if at least one of the above parameters is improved while one or more of the other parameters is not, as long as the optimized sequence leads to an improvement of the transgene, such as an improved expression of the transgene in vivo.

The LAL moiety of the nucleic acid molecule of the invention preferably has at least 85 percent, more preferably at least 90 percent, and even more preferably at least 92 percent identity or at least 94 percent identity, in particular at least 95 percent identity, for example at least 96, 97, 98, 99 or 100 percent identity to the nucleotide sequence of SEQ ID NO: 15 to SEQ ID NO:17, preferably to the nucleotide sequence of SEQ ID NO:16 or SEQ ID NO:17, which are sequences optimized for transgene expression in vivo.

The nucleic acid molecule of the invention encodes a chimeric functional LAL protein, in which a functional LAL moiety as described above is fused to an heterologous signal peptide. By “heterologous signal peptide” is meant a peptide of another protein than LAL protein. Thus, the nucleic acid molecule therefore encodes a chimeric LAL polypeptide comprising a signal peptide from another protein than a LAL, operably linked to a LAL polypeptide.

In a particular embodiment, in the encoded chimeric LAL polypeptide, the endogenous (or natural) signal peptide of a LAL polypeptide is replaced with an heterologous signal peptide, i.e. a signal peptide of another protein. The encoded chimeric polypeptide is thus a functional LAL protein wherein the amino acid sequence corresponding to the natural signal peptide of LAL (such as the first 21 to 27 amino acid residues, in particular the first 23 amino acid residues, at the N-terminus of hLAL of SEQ ID NO:8) is replaced by the amino acid sequence of a signal peptide of a different protein. From the foregoing, as compared to a wild-type LAL polypeptide, the endogenous signal peptide of wild-type LAL is replaced with an heterologous signal peptide, i.e. a signal peptide derived from a protein different from LAL.

In a particular embodiment, the heterologous signal peptide fused to the LAL protein increases the secretion of the resulting chimeric LAL polypeptide as compared to the corresponding LAL polypeptide comprising its natural signal peptide. The relative proportion of LAL that is secreted from the cell can be routinely determined by methods known in the art and described in the examples. Secreted proteins can be detected by directly measuring the protein itself (e.g., by Western blot) or by protein activity assays (e.g., enzyme assays) in cell culture medium, serum, milk, etc.

In another particular embodiment, the heterologous signal peptide fused to the LAL protein increases the secretion and expression of the resulting chimeric LAL polypeptide as compared to the corresponding LAL polypeptide comprising its natural signal peptide, in particular without decreasing its enzymatic activity, nor its ability to cross-correct LAL deficient cells.

The signal peptides workable in the present invention include, without limitation, amino acids 1-25 from iduronate-2-sulphatase (SEQ ID NO:3), amino acids 1-18 from chymotrypsinogen B2 (SEQ ID NO:4) and amino acids 1-22 from protease Cl inhibitor (SEQ ID NO:5). The inventors have surprisingly shown that the signal peptides of SEQ ID NO:3 to SEQ ID NO:5, allow higher secretion and expression of the chimeric LAL protein when compared to LAL comprising its natural signal peptide, or to a chimeric LAL protein comprising other heterologous signal peptides such as the signal peptide of hAAT. Said signal peptides are shown to improve LAL secretion and expression while preserving its enzymatic activity and its ability to cross-correct LAL deficient cells. Thus, the nucleic acid molecule of the invention comprises a sequence encoding a signal peptide having an amino acid sequence selected in the group consisting of SEQ ID NO:3 to 5 (otherwise referred to herein as an “alternative signal peptide”).

In addition, the signal peptide moiety of the chimeric LAL protein encoded by the nucleic acid molecule of the invention may comprise from 1 to 5, in particular from 1 to 4, in particular from 1 to 3, more particularly from 1 to 2, in particular 1 amino acid deletion(s), insertion(s) or substitution(s) as compared to the sequences shown in SEQ ID NO:3 to 5, as long as the resulting sequence corresponds to a functional signal peptide, i.e. a signal peptide that allows secretion of a LAL protein which is higher than the secretion observed with the natural signal peptide of LAL. In a particular embodiment, the signal peptide moiety sequence consists of a sequence selected in the group consisting of SEQ ID NO:3 to 5.

Those skilled in the art will further understand that the chimeric LAL polypeptide can contain additional amino acids, e. g., as a result of manipulations of the nucleic acid construct such as the addition of a restriction site, as long as these additional amino acids do not render the signal peptide or the LAL polypeptide non-functional. The additional amino acids can be cleaved or can be retained by the mature polypeptide as long as retention does not result in a non-functional polypeptide.

In a particular embodiment, the nucleic acid molecule of the invention comprises the nucleic acid sequence of SEQ ID NO:11 (encoding the signal peptide of SEQ ID NO:3), of SEQ ID NO:12 (encoding the signal peptide of SEQ ID NO:4) or of SEQ ID NO:13 (encoding the signal peptide of SEQ ID NO:5).

Furthermore, according to a particular embodiment of the invention, the nucleotide sequence corresponding to the alternative signal peptide may be an optimized sequence.

For example, SEQ ID NO:18-20 correspond to optimized sequences encoding the signal peptide of SEQ ID NO:5. Preferably, the sequence encoding the signal peptide is SEQ ID NO:19 or SEQ ID NO:20.

The nucleic acid molecule of the invention encodes a functional chimeric LAL polypeptide, i.e. it encodes for a chimeric LAL polypeptide that, when expressed, has the functionality of wild-type LAL protein, in particular of hLAL. As defined above, the functionality of wild-type LAL is to hydrolyse cholesteryl esters and triglyceride, to liberate fatty acids and cholesterol. The chimeric functional LAL polypeptide encoded by the nucleic acid of the invention may have a hydrolysing activity of at least 50%, 60%, 70%, 80%, 90%, 95%, 99%, or at least 100% as compared to a wild-type LAL polypeptide, in particular as compared to the human wild-type LAL, more particularly as compared to the hLAL encoded by the nucleic acid sequence of SEQ ID NO:10 (i.e. the LAL polypeptide having the amino acid sequence of SEQ ID NO:8). The activity of the chimeric LAL protein encoded by the nucleic acid of the invention may even be of more than 100%, such as of more than 110%, 120%, 130%, 140%, or even more than 150% of the activity of a wild-type LAL polypeptide, in particular of the human wild-type LAL, more particularly of the hLAL encoded by the nucleic acid sequence of SEQ ID NO:10 (i.e. the LAL polypeptide having the amino acid sequence of SEQ ID NO:8).

In a particular embodiment, the nucleic acid molecule of the invention is a nucleotide sequence optimized to improve the expression of the chimeric LAL protein. In a particular embodiment, the nucleic acid molecule of the invention comprises an optimized signal peptide moiety coding sequence and/or an optimized LAL moiety coding sequence.

In a particular embodiment, the nucleic acid molecule of the invention comprises or consists of a nucleotide sequence resulting from the combination of:
a signal peptide moiety coding sequence selected in the group consisting of SEQ ID NO:11 to 13; and
a LAL moiety coding sequence selected in the group consisting of SEQ ID NO:14 to 17.

In a particular embodiment, the nucleic acid molecule of the invention comprises or consists of a nucleotide sequence resulting from the combination of:
a signal peptide moiety coding sequence of SEQ ID NO:13; and
a LAL moiety coding sequence selected in the group consisting of SEQ ID NO:14 to 17.

In a particular embodiment, the nucleic acid molecule of the invention comprises or consists of a nucleotide sequence resulting from the combination of:
an optimized signal peptide moiety coding sequence selected in the group consisting of SEQ ID NO:18 to 20, preferably SEQ ID NO:19 or SEQ ID NO:20; and
a LAL moiety coding sequence selected in the group consisting of SEQ ID NO:14 to 17.

In a particular embodiment, the nucleic acid molecule of the invention comprises or consists of a nucleotide sequence resulting from the combination of:
a signal peptide moiety coding sequence selected in the group consisting of SEQ ID NO:11 to 13, preferably SEQ ID NO:13; and
a LAL moiety coding sequence of SEQ ID NO:14.

In a particular embodiment, the nucleic acid molecule of the invention comprises or consists of a nucleotide sequence resulting from the combination of:
a signal peptide moiety coding sequence selected in the group consisting of SEQ ID NO:11 to 13, preferably SEQ ID NO:13; and
an optimized LAL moiety coding sequence of SEQ ID NO:15.

In a particular embodiment, the nucleic acid molecule of the invention comprises or consists of a nucleotide sequence resulting from the combination of:
a signal peptide moiety coding sequence selected in the group consisting of SEQ ID NO:11 to 13, preferably SEQ ID NO:13; and
an optimized LAL moiety coding sequence of SEQ ID NO:16.

In a particular embodiment, the nucleic acid molecule of the invention comprises or consists of a nucleotide sequence resulting from the combination of:
a signal peptide moiety coding sequence selected in the group consisting of SEQ ID NO:11 to 13, preferably SEQ ID NO:13; and
an optimized LAL moiety coding sequence of SEQ ID NO:17.

In a particular embodiment, the nucleic acid molecule of the invention comprises or consists of a nucleotide sequence resulting from the combination of:
an optimized signal peptide moiety coding sequence selected in the group consisting of SEQ ID NO:18 to 20, preferably SEQ ID NO:19 or SEQ ID NO:20; and
a LAL moiety coding sequence of SEQ ID NO:14.

In a particular embodiment, the nucleic acid molecule of the invention comprises or consists of a nucleotide sequence resulting from the combination of:
an optimized signal peptide moiety coding sequence selected in the group consisting of SEQ ID NO:18 to 20, preferably SEQ ID NO:19 or SEQ ID NO:20; and
an optimized LAL moiety coding sequence of SEQ ID NO:15.

In a particular embodiment, the nucleic acid molecule of the invention comprises or consists of a nucleotide sequence resulting from the combination of:
a signal peptide moiety coding sequence selected in the group consisting of SEQ ID NO:18 to 20, preferably SEQ ID NO:19 or SEQ ID NO:20; and
an optimized LAL moiety coding sequence of SEQ ID NO:16.

In a particular embodiment, the nucleic acid molecule of the invention comprises or consists of a nucleotide sequence resulting from the combination of:
a signal peptide moiety coding sequence selected in the group consisting of SEQ ID NO:18 to 20, preferably SEQ ID NO:19 or SEQ ID NO:20; and
an optimized LAL moiety coding sequence of SEQ ID NO:17.

In a particular embodiment, the nucleic acid molecule encodes a functional chimeric LAL protein comprising a functional LAL moiety as described above and at least one signal peptide moiety as described above, such as two, three, four or five signal peptide moieties as described above.

In a particular embodiment, the nucleic acid molecule of the invention comprises or consists of a nucleotide sequence of SEQ ID NO:21, SEQ ID NO:22 or SEQ ID NO:23, or of a nucleotide sequence having at least 80%, at least 85%, at least 90%, 91%, 92%, 93%, 94%, 95%, 96%, 97%, 98% or at least 99% identity to the sequence of SEQ ID NO:21, SEQ ID NO:22 or SEQ ID NO:23. Preferably, the nucleic acid molecule of the invention comprises or consists of a nucleotide sequence of SEQ ID NO:22 or SEQ ID NO:23, or of a nucleotide sequence having at least 80%, at least 85%, at least 90%, 91%, 92%, 93%, 94%, 95%, 96%, 97%, 98% or at least 99% identity to the sequence of SEQ ID NO:22 or SEQ ID NO:23.

Nucleic acid Construct

The invention also relates to a nucleic acid construct comprising a nucleic acid molecule of the invention.

The nucleic acid construct may correspond to an expression cassette comprising the nucleic acid sequence of the invention, operably linked to one or more expression control sequences and/or other sequences improving the expression of a transgene and/or sequences enhancing the secretion of the encoded protein and/or sequences enhancing the uptake of the encoded protein. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers (such as cis-regulatory modules (CRMs)), introns, kozac sequence, polyA signals, etc.

In particular, the expression cassette may include a promoter. The promoter may be an ubiquitous or tissue-specific promoter, in particular a promoter able to promote expression in cells or tissues in which expression of LAL is desirable such as in cells or tissues in which LAL expression is desirable in LAL-deficient patients. In a particular embodiment, the promoter is a liver-specific promoter such as the alpha-1 antitrypsin promoter (hAAT), the transthyretin promoter, the albumin promoter, the thyroxine-binding globulin (TBG) promoter, the LSP promoter (comprising a thyroid hormone-binding globulin promoter sequence, two copies of an alpha1-microglobulin/bikunin enhancer sequence, and a leader sequence—34.Ill, C. R., et al. (1997). Optimization of the human factor VIII complementary DNA expression plasmid for gene therapy of hemophilia A. Blood Coag. Fibrinol. 8: S23-S30.), etc. Other useful liver-specific promoters are known in the art, for example those listed in the Liver Specific Gene Promoter Database compiled the Cold Spring Harbor Laboratory (http://rulai.cshl.edu/LSPD/). A preferred promoter in the context of the invention is the hAAT promoter.

Other tissue-specific or non-tissue-specific promoters may be useful in the practice of the invention. For example, the expression cassette may include a tissue-specific promoter, which is a promoter different from a liver specific promoter.

In another particular embodiment, the promoter is an erythroid-specific promoter, for the expression of the LAL polypeptide from cells of the erythroid lineage. In a particular embodiment, the promoter is suitable for transgene expression into hematopoietic stem cells. As “erythroid-specific promoter”, one can cite without limitation: a human beta-globin promoter; the promoter region of the human erythroid genes Kruppel-like factor 1 (KLF1); the spectrin alpha gene (SPTA1) promoter; the GATA-1 gene promoter; the BVL-1-like VL30 promoter; the ankyrin-1 promoter; the pyruvate kinase erythroid-specific promoter; or the promoter of glycophorin A (GPA) gene or glycophorin B (GPG) gene. Said erythroid promoter may be associated to an enhancer such as an enhancer specific to erythroid lineage. Enhancers specific to erythroid lineage include without limitation: β-globin HS2, α-globin HS40, GATA-1, ARE and ALAS2 intron 8 enhancers.

In another embodiment, the promoter is an ubiquitous promoter. Representative ubiquitous promoters include the cytomegalovirus enhancer/chicken beta actin (CAG) promoter, the cytomegalovirus enhancer/promoter (CMV), the PGK promoter, the SV40 early promoter, etc.

In addition, the promoter may also be an endogenous promoter such as the albumin promoter or the LAL promoter.

In a particular embodiment, the promoter is associated to an enhancer sequence, such as cis-regulatory modules (CRMs) or an artificial enhancer sequence. For example, the promoter may be associated to an enhancer sequence such as the human ApoE control region (or Human apolipoprotein E/C-I gene locus, hepatic control region HCR-1—Genbank accession No. U32510). In a particular embodiment, an enhancer sequence such as the ApoE sequence is associated to a liver-specific promoter such as those listed above, and in particular such as the hAAT promoter. Other CRMs useful in the practice of the present invention include those described in Rincon et al., Mol Ther. 2015 Jan;23(1):43-52, Chuah et al., Mol Ther. 2014 Sep;22(9):1605-13 or Nair et al., Blood. 2014 May 15;123(20):3195-9.

In a particular embodiment, the nucleic acid construct comprising the nucleic acid molecule of the invention is an expression cassette which comprises said nucleic acid molecule operably linked to a promoter, wherein said nucleic acid construct optionally further comprises an intron and/or post-transcriptional regulatory sequence(s).

By “post-transcriptional regulatory sequence” is meant any sequence able to regulate the expression via a post-transcriptional pathway, such as any sequence acting on mRNA stability and trafficking. Post-transcriptional regulatory sequences include for example the polyadenylation signal, 3′ and 5′ UTR sequences or miRNA binding sites.

In another particular embodiment, the nucleic acid construct comprises an intron, in particular an intron placed between the promoter and the LAL coding sequence. An intron may be introduced to increase mRNA stability and the production of the protein. In a further embodiment, the nucleic acid construct comprises a human beta globin b2 (or HBB2) intron, a coagulation factor IX (FIX) intron, a SV40 intron or a chicken beta-globin intron. In another further embodiment, the nucleic acid construct of the invention contains a modified intron (in particular a modified HBB2 or FIX intron) designed to decrease the number of, or even totally remove, alternative open reading frames (ARFs) found in said intron. Preferably, ARFs are removed whose length spans over 50 bp and have a stop codon in frame with a start codon. ARFs may be removed by modifying the sequence of the intron. For example, modification may be carried out by way of nucleotide substitution, insertion or deletion, preferably by nucleotide substitution. As an illustration, one or more nucleotides, in particular one nucleotide, in an ATG or GTG start codon present in the sequence of the intron of interest may be replaced resulting in a non-start codon. For example, an ATG or a GTG may be replaced by a CTG, which is not a start codon, within the sequence of the intron of interest.

In a particular embodiment, the nucleic acid construct of the invention is an expression cassette comprising, in the 5′ to 3′ orientation, a promoter optionally preceded by an enhancer, a LAL coding sequence (such as the chimeric LAL coding sequence, or the chimeric and optimized LAL coding sequence as described above), and a polyadenylation signal (such as the bovine growth hormone polyadenylation signal, the SV40 polyadenylation signal, or another naturally occurring or artificial polyadenylation signal). In particular embodiments, the expression cassette contains the coding sequence resulting from the combinations of signal peptide coding sequence and LAL moiety coding sequence as described above.

In designing the nucleic acid construct of the invention, one skilled in the art will take care of respecting the size limit of the vector used for delivering said construct to a cell or organ. In particular, one skilled in the art knows that a major limitation of AAV vector is its cargo capacity which may vary from one AAV serotype to another but is thought to be limited to around the size of parental viral genome. For example, 5 kb, is the maximum size usually thought to be packaged into an AAV8 capsid (Wu Z. et al., Mol Ther., 2010, 18(1): 80-86; Lai Y. et al., Mol Ther., 2010, 18(1): 75-79; Wang Y. et al., Hum Gene Ther Methods, 2012, 23(4): 225-33). Accordingly, those skilled in the art will take care in practicing the present invention to select the components of the nucleic acid construct of the invention so that the resulting nucleic acid sequence, including sequences coding AAV 5′- and 3′-ITRs to preferably not exceed 110% of the cargo capacity of the AAV vector implemented, in particular to preferably not exceed 5.5 kb.

### Vector

The invention also relates to a vector comprising a nucleic acid molecule or construct as disclosed herein.

In particular, the vector of the invention is a vector suitable for protein expression, preferably for use in gene therapy. In one embodiment, the vector is a plasmid vector. In another embodiment, the vector is a nanoparticle containing a nucleic acid molecule of the invention, in particular a messenger RNA encoding the chimeric LAL polypeptide of the invention. In another embodiment, the vector is a system based on transposons, allowing integration of the nucleic acid molecule or construct of the invention in the genome of the target cell, such as the hyperactive Sleeping Beauty (SB100×) transposon system (Mates et al. 2009).

In another embodiment, the vector is a viral vector suitable for gene therapy, targeting any cell of interest. For example, the vector may target all cell lineages (ubiquitous vector) or may target the liver tissue, the microglia or hematopoietic stem cells such as cells of the erythroid lineage (such as erythrocytes). In this case, the nucleic acid construct of the invention also contains sequences suitable for producing an efficient viral vector, as is well known in the art. In a particular embodiment, the viral vector is derived from an integrating virus. In particular, the viral vector may be derived from a retrovirus or a lentivirus, for example lentiviral vector derived from the human immunodeficiency virus (HIV). In a further particular embodiment, the viral vector is an AAV vector, such as an AAV vector suitable for transducing liver tissues or cells, more particularly an AAV-1, -2 and AAV-2 variants (such as the quadruple-mutant capsid optimized AAV-2 comprising an engineered capsid with Y44+500+730F+T491V changes, disclosed in Ling et al., 2016 Jul 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026), -7, -8, -9, -10 such as -cy10 and -rh10, -rh74, -dj, Anc80, LK03, AAV2i8, porcine AAV serotypes such as AAVpo4 and AAVpo6, etc., vector or a retroviral vector such as a lentiviral vector and an alpha-retrovirus. As is known in the art, depending on the specific viral vector considered for use, additional suitable sequences will be introduced in the nucleic acid construct of the invention for obtaining a functional viral vector. Suitable sequences include AAV ITRs for an AAV vector, or LTRs for lentiviral vectors. As such, the invention also relates to an expression cassette as described above, flanked by an ITR or an LTR on each side.

Advantages of viral vectors are discussed in the following part of this disclosure. Viral vectors are preferred for delivering the nucleic acid molecule or construct of the invention, such as a retroviral vector, for example a lentiviral vector, or a non-pathogenic parvovirus, more preferably an AAV vector. The human parvovirus Adeno-Associated Virus (AAV) is a dependovirus that is naturally defective for replication which is able to integrate into the genome of the infected cell to establish a latent infection.

The last property appears to be unique among mammalian viruses because the integration occurs at a specific site in the human genome, called AAVS1, located on chromosome 19 (19q13.3-qter). Therefore, AAV vectors have arisen considerable interest as a potential vectors for human gene therapy. Among the favorable properties of the virus are its lack of association with any human disease, its ability to infect both dividing and non-dividing cells, and the wide range of cell lines derived from different tissues that can be infected.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026), -7, -8, -9, -10 such as cy10 and -rh10, -rh74, -dj, Anc80, LK03, AAV2i8, porcine AAV serotypes such as AAVpo4 and AAVpo6, and tyrosine, lysine and serine capsid mutants of the AAV serotypes, etc. In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus. Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. AAV-based recombinant vectors lacking the Rep protein integrate with low efficacy into the host's genome and are mainly present as stable circular episomes that can persist for years in the target cells.

Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Accordingly, the present invention relates to an AAV vector comprising the nucleic acid molecule or construct of the invention. In the context of the present invention, the AAV vector comprises an AAV capsid able to transduce the target cells of interest, in particular hepatocytes or cells of the erythroid lineage. According to a particular embodiment, the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026), -7, -8, -9, -10 such as -cy10 and -rh10, -rh74, -dj, Anc80, LK03, AAV2i8, porcine AAV such as AAVpo4 and AAVpo6, and tyrosine, lysine and serine capsid mutants of a AAV serotypes, etc., serotype. In a particular embodiment, the AAV vector is of the AAV8, AAV9, AAVrh74 or AAV2i8 serotype (i.e. the AAV vector has a capsid of the AAV8, AAV9, AAVrh74 or AAV2i8 serotype). In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, and whose capsid is derived from another serotype. For example, the genome of the pseudotyped vector may have a capsid derived from the AAV8, AAV9, AAVrh74 or AAV2i8 serotype, and its genome may be derived from and different serotype.

In another specific embodiment, wherein the vector is for use in delivering the transgene to liver cells, the AAV vector may be selected, among others, in the group consisting of AAV5, AAV6, AAV-DJ, AAV8, AAV9, AAV-LK03, AAV-Anc80 and AAV3B.

In another embodiment, the capsid is a modified capsid. In the context of the present invention, a “modified capsid” may be a chimeric capsid or capsid comprising one or more variant VP capsid proteins derived from one or more wild-type AAV VP capsid proteins. In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. Examples of such chimeric AAV vectors useful to transduce liver cells are described in Shen et al., Molecular Therapy, 2007 and in Tenney et al., Virology, 2014. For example a chimeric AAV vector can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1 , RHM15-2, RHM15-3/RHM15-5, RHM15-4 and RHM15-6 capsid variants, which present a high liver tropism.

In another embodiment, the modified capsid can be derived also from capsid modifications inserted by error prone PCR and/or peptide insertion (e.g. as described in Bartel et al., 2011). In addition, capsid variants may include single amino acid changes such as tyrosine mutants (e.g. as described in Zhong et al., 2008)

In addition, the genome of the AAV vector may either be a single stranded or self-complementary double-stranded genome (McCarty et al., Gene Therapy, 2003). Self-complementary double-stranded AAV vectors are generated by deleting the terminal resolution site (trs) from one of the AAV terminal repeats. These modified vectors, whose replicating genome is half the length of the wild type AAV genome have the tendency to package DNA dimers. In a preferred embodiment, the AAV vector implemented in the practice of the present invention has a single stranded genome, and further preferably comprises an AAV2, AAV6, AAV-DJ, AAV8 or AAV9 capsid. Preferably, the AAV vector comprises an AAV6 capsid or an AAV6-derived capsid.

In a particular embodiment, the invention relates to an AAV vector or a lentiviral vector comprising, in a single-stranded or double-stranded, self-complementary genome (e.g. a single-stranded genome), the nucleic acid molecule of the invention. In a further particular embodiment, said nucleic acid molecule is operably linked to a promoter, especially an ubiquitous, liver-specific or erythroid-specific promoter as described above. In a further particular embodiment, the nucleic acid construct comprised into the genome of the viral vector of the invention further comprises an intron as described above, such as an intron placed between the promoter and the nucleic acid sequence encoding the LAL coding sequence

As an alternative to the use of expression vector administered in vivo, cells can be infected ex vivo or in vitro with the nucleic acid molecule of the invention. These cells can then be administered to the individual in need thereof and play the role of gene delivery systems (see for example patent application WO19138082). For example, the use of an ex vivo or in vitro generated hematopoietic stem cell as described herein by transgene targeted integration under the control of an active endogenous promoter advantageously minimizes the risk of insertional mutagenesis and oncogene transactivation associated with the use of semi-random integrating vectors and the risk of gene transactivation, as no exogenous promoter/enhancer elements are required for transgene expression and inserted in the genome. This method is highly advantageous to the individual in need thereof, as most of the current treatments for the diseases considered herein consist in frequent injections of the therapeutic protein, which is demanding, expensive, not curative on the long term and leads to the development of anti-protein neutralizing antibodies in a high percentage of the treated patients.

In a particular embodiment, the vector is a vector suitable for integrating the nucleic acid molecule of the invention into the genome of cells, in particular cells of the erythroid lineage such as hematopoietic stem cells. As used herein, the vector can refer to a nucleic acid vector (e.g., a plasmid or recombinant viral genome) or a viral vector (e.g., an rAAV particle comprising a recombinant genome).

Suitable vectors for integrating the nucleic acid molecule of the invention into the genome of cells of the erythroid lineage such as hematopoietic stem cells include lentiviral vector and AAV vectors. In particular, the lentiviral vector is replication-defective, e.g., does not comprise one or more genes required for viral replication. Exemplary AAV vectors that may be used include an AAV2 vector, a modified AAV2 vector, an AAV3 vector, a modified AAV3 vector, an AAV6 vector, a modified AAV6 vector, an AAV8 vector and an AAV9 vector. In a particular embodiment, a CRISPR/Cas9-mediated integration of the nucleic molecule of the invention may be carried our using a lentiviral vector or an AAV vector.

Auxiliary vector(s) may be used for providing to the cell a site-directed genetic engineering system, enabling the integration of the nucleic acid molecule of the invention into the cell genome.

### Chimeric LAL Protein

In another aspect, the invention provides a chimeric LAL polypeptide encoded by the nucleic acid molecule of the invention.

The chimeric LAL polypeptide of the invention is a functional chimeric LAL protein., i.e. it has the functionality of wild-type LAL protein, in particular of human LAL (hLAL). As defined above, the functionality of wild-type LAL is to hydrolyse cholesteryl esters and triglyceride, to liberate fatty acids and cholesterol. The chimeric LAL protein of the invention may have a hydrolysing activity of at least 50%, 60%, 70%, 80%, 90%, 95%, 99%, or at least 100% as compared to the wild-type human LAL polypeptide. The activity of the chimeric LAL protein of the invention may even be of more than 100%, such as of more than 110%, 120%, 130%, 140%, or even more than 150% of the activity of the wild-type human LAL polypeptide.

As described above, the chimeric LAL polypeptide of the invention comprises an heterologous signal peptide moiety and a functional LAL moiety.

The “functional LAL moiety” refers to any LAL polypeptide that has the functionality of wild-type LAL protein, in particular of human LAL (hLAL). According to a particular embodiment, the “functional LAL moiety” is a human functional LAL polypeptide. As defined above, the term “functional LAL moiety” encompasses mature and precursor LAL, as well as modified or mutated by insertion(s), deletion (s) and/or substitution(s) LAL proteins or fragments thereof that are functional derivatives of LAL, i.e. that retain biological function of LAL. Furthermore, the chimeric LAL polypeptide may comprise a LAL moiety that is a functional, truncated form of LAL.

The functional LAL moiety may be a “precursor form of LAL”, i.e. a LAL polypeptide that comprises its natural signal peptide. For example, the functional LAL moiety may have the sequence of SEQ ID NO:8 (399 amino acid residues long) is the precursor form of human LAL (hLAL). In a particular embodiment, the functional LAL moiety comprises or consists of SEQ ID NO:8, or comprises or consists of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:8.

In a preferred embodiment, the functional LAL moiety is a mature form of LAL, in particular of hLAL, i.e. corresponds to the precursor form of LAL polypeptide as defined above, but devoid of its natural signal peptide. In a particular embodiment, the functional LAL moiety is a functional human LAL moiety devoid of its natural signal peptide. The signal peptide of human LAL is any of the first 21 to 27 amino acid residues at the N-terminus of precursor hLAL of SEQ ID NO:8. After cleavage of the signal peptide, a mature form of hLAL can be 372 to 378 amino acid residues long, depending on the length of the signal peptide sequence cleaved off from the 399 amino acid residues long hLAL. Thus, the mature form of hLAL without the signal peptide can include hLAL having 378 amino acid residues (i.e., from Ser22 to Gln399 of SEQ ID NO:8), 377 amino acid residues (i.e., from Gly23 to Gln399 of SEQ ID NO:8), 376 amino acid residues (i.e., from Gly24 to Gln399 of SEQ ID NO:8), 375 amino acid residues (i.e., from Lys25 to Gln399 of SEQ ID NO:8), 374 amino acid residues (i.e., from Leu26 to Gln399 of SEQ ID NO:8), 373 amino acid residues (i.e., from Thr27 to Gln399 of SEQ ID NO:8), or 372 amino acid residues (i.e., from Ala28 to Gln399 of SEQ ID NO:8).

In a particular embodiment, the signal peptide of hLAL corresponds to the first 23 amino acid residues at the N-terminus of the precursor hLAL of SEQ ID NO:8. According to this embodiment, the signal peptide of hLAL is as shown in SEQ ID NO:1. Thus, in a particular embodiment, the functional LAL moiety is a functional human LAL moiety devoid of the natural signal peptide shown in SEQ ID NO:1.

According to a particular embodiment, the mature form of hLAL (without the signal peptide) corresponds to a sequence of 376 amino acid residues as referred in SEQ ID NO:9.

In a particular embodiment, the functional LAL moiety comprises or consists of SEQ ID NO:9, or comprises or consists of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:9.

In a particular embodiment, the functional LAL moiety comprises or consists of SEQ ID NO:9, or a functional variant thereof comprising from 1 to 50, in particular from 1 to 40, in particular from 1 to 30, in particular from 1 to 20, in particular from 1 to 10 or from 1 to 5 amino acid substitutions as compared to the sequence shown in SEQ ID NO:9, such as 1, 2, 3, 4 or 5 amino acid substitutions.

As defined above, an “heterologous signal peptide” is a peptide of another protein than LAL protein. Thus, the chimeric LAL polypeptide of the invention comprises a signal peptide from another protein than a LAL, operably linked to a LAL polypeptide.

In a particular embodiment, the endogenous (or natural) signal peptide of a LAL polypeptide is replaced with an heterologous signal peptide, i.e. a signal peptide of another protein. The chimeric polypeptide is thus a functional LAL protein wherein the amino acid sequence corresponding to the natural signal peptide of LAL (such as the first 21 to 27 amino acid residues, in particular the first 23 amino acid residues, at the N-terminus of hLAL of SEQ ID NO:8) is replaced by the amino acid sequence of a signal peptide of a different protein. From the foregoing, as compared to a wild-type LAL polypeptide, the endogenous signal peptide of wild-type LAL is replaced with an heterologous signal peptide, i.e. a signal peptide derived from a protein different from LAL.

In a particular embodiment, the heterologous signal peptide fused to the LAL moiety increases the secretion and expression of the resulting chimeric LAL polypeptide as compared to the corresponding LAL polypeptide comprising its natural signal peptide, in particular without decreasing its enzymatic activity, nor its ability to cross-correct LAL deficient cells.

The signal peptides workable in the present invention include amino acids 1-25 from iduronate-2-sulphatase (SEQ ID NO:3), amino acids 1-18 from chymotrypsinogen B2 (SEQ ID NO:4) and amino acids 1-22 from protease Cl inhibitor (SEQ ID NO:5). The inventors have surprisingly shown that the signal peptides of SEQ ID NO:3 to SEQ ID NO:5, allow higher secretion and expression of the chimeric LAL protein when compared to the LAL comprising its natural signal peptide, or to a chimeric LAL protein comprising other heterologous signal peptides such as the signal peptide of hAAT. Said signal peptides are shown to improve LAL secretion and expression while preserving its enzymatic activity and its ability to cross-correct LAL deficient cells. Thus, the chimeric LAL protein of the invention comprises a sequence encoding a signal peptide having an amino acid sequence selected in the group consisting of SEQ ID NO:3 to 5 (otherwise referred to herein as an “alternative signal peptide”).

In addition, the signal peptide moiety of the chimeric LAL protein of the invention may comprise from 1 to 5, in particular from 1 to 4, in particular from 1 to 3, more particularly from 1 to 2, in particular 1 amino acid deletion(s), insertion(s) or substitution(s) as compared to the sequences shown in SEQ ID NO:3 to 5, as long as the resulting sequence corresponds to a functional signal peptide, i.e. a signal peptide to that allows secretion of a LAL protein. In a particular embodiment, the signal peptide moiety sequence consists of a sequence selected in the group consisting of SEQ ID NO:3 to 5.

Those skilled in the art will further understand that the chimeric LAL polypeptide can contain additional amino acids, e. g., as a result of manipulations of the nucleic acid construct such as the addition of a restriction site, as long as these additional amino acids do not render the signal peptide or the LAL polypeptide non-functional. The additional amino acids can be cleaved or can be retained by the mature polypeptide as long as retention does not result in a non-functional polypeptide.

In particular embodiment, the functional chimeric LAL protein of the invention comprises or consists of an amino acid sequence resulting from the combination of:
a signal peptide moiety sequence selected in the group consisting of SEQ ID NO:3 to 5, preferably SEQ ID NO:5; and
a functional LAL moiety sequence consisting of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the sequence as shown in SEQ ID NO:9.

In particular embodiment, the functional chimeric LAL protein of the invention comprises or consists of an amino acid sequence resulting from the combination of:
a signal peptide moiety sequence selected in the group consisting of SEQ ID NO:3 to 5, preferably SEQ ID NO:5; and
a functional LAL moiety sequence consisting of the sequence as shown in SEQ ID NO:9.

In a particular embodiment, the functional chimeric LAL protein comprises a functional LAL moiety as described above and at least one signal peptide moiety as described above, such as two, three, four or five signal peptide moieties as described above.

In a particular embodiment, the functional chimeric LAL protein of the invention comprises or consists of the amino acid sequence of SEQ ID NO: 30 to 32, preferably SEQ ID NO:32, or a variant thereof having from 1 to 40, in particular from 1 to 20, in particular from 1 to 10 or from 1 to 5 amino acid substitutions as compared to the sequence shown in SEQ ID NO:30 to 32, preferably SEQ ID NO:32, such as 1, 2, 3, 4 or 5 amino acid substitutions.

In a particular embodiment, the functional chimeric LAL protein of the invention does not comprise a moiety consisting of serum albumin, such as human serum albumin.

### Cell

The invention also relates to a cell that is transformed with a nucleic acid molecule or construct of the invention as is the case for ex vivo gene therapy. Thus, the invention relates to an isolated cell, for example a liver cell, that comprises the nucleic acid molecule, the nucleic acid construct or the vector of the invention. Cells of the invention may be delivered to the subject in need thereof, such as LAL-deficient patient, by any appropriate administration route such as via injection in the bloodstream of said subject.

In a particular embodiment, the invention involves introducing the nucleic acid of the invention into cells such as hematopoietic stem cells or liver cells of the subject to be treated, and administering said transformed cells into which the nucleic acid molecule has been introduced to the subject. Advantageously, this embodiment is useful for secreting LAL from said cells. In a particular embodiment, the cells are cells from the patient to be treated.

In a preferred embodiment, the cell is an hematopoietic stem cell. Hematopoietic stem cells (HSC) are pluripotent stem cells capable of self-renewal and are characterized by their ability to give rise under permissive conditions to all cell types of the hematopoietic system. Hematopoietic stem cells are not totipotent cells, i.e. they are not capable of developing into a complete organism. Advantageously, erythroid cells are the most abundant hematopoietic progeny (˜2×10 new erythrocytes per day) and can secrete relevant amounts of therapeutic proteins.

In a particular embodiment, the invention relates to genetically modified hematopoietic stem cells that, when differentiated towards the erythroid lineage, are able to produce the LAL chimeric therapeutic protein encoded by the nucleic acid molecule of the invention. Thus, another object of the present invention relates to a genetically modified hematopoietic stem cell comprising, in its genome, the nucleic acid molecule of the invention.

In a particular embodiment, the genetically modified hematopoietic stem cell comprises, in the intergenic regions flanking at least one globin gene comprised in the genome thereof, the nucleic acid molecule of the invention, placed under the control of the endogenous promoter of said globin gene.

The genetically modified HSC and the method for generating said cell may be as described in patent application WO19138082.

In a particular embodiment, the HSC according to the invention is derived from an embryonic stem cell, in particular from a human embryonic stem cell, and is thus an embryonic hematopoietic stem cell. Embryonic stem cells (ESCs) are stem cells derived from the undifferentiated inner mass cells of an embryo and capable of self-renewal. Under permissive conditions, these pluripotent stem cells are capable of differentiating in any one of the more than 220 cell types in the adult body. Embryonic stem cells can for example be obtained according to the method indicated in Young Chung et al. (Cell Stem Cell 2, 2008 Feb. 7;2(2): 113-7.

In another particular embodiment, a hematopoietic stem cell according to the invention is an induced pluripotent stem cell, more particularly a human induced pluripotent stem cell (hiPSCs). Thus, according to a particular embodiment, hematopoietic stem cells as described herein are hematopoietic induced pluripotent stem cells.

In a particular embodiment, the initial population of hematopoietic stem cells and/or blood cells may be autologous. “Autologous” refers to deriving from or originating in the same patient or individual. An “autologous transplant” refers to the harvesting and reinfusion or transplant of a subject's own cells or organs. Exclusive or supplemental use of autologous cells can eliminate or reduce many adverse effects of administration of the cells back to the host, particular graft versus host reaction. In this case, the hematopoietic stem cells were collected from the said individual, genetically modified ex vivo or in vitro to integrate the nucleic acid molecule of the invention and administered to the same individual.

In a particular embodiment, the initial population of hematopoietic stem cells and/or blood cells may be derived from an allogeneic donor or from a plurality of allogeneic donors. The donors may be related or unrelated to each other, and in the transplant setting, related or unrelated to the recipient (or individual).

The stem cells to be modified may accordingly be exogenous to the individual in need of therapy. In situations of administration of modified stem cells of exogenous origin, the said stem cells may be syngeneic, allogeneic, xenogeneic, or a mixture thereof.

In another embodiment, the stem cell described herein is a mammalian cell, in particular a human cell.

Another object of the present invention relates to a blood cell originating from a genetically modified hematopoietic stem cell as described herein. Accordingly, in a particular embodiment, the said blood cell is selected from the group consisting of megakaryocytes, thrombocytes, erythrocytes, mast cells, myeloblasts, basophils, neutrophils, eosinophils, monocytes, macrophages, natural killer cells, small lymphocytes, T lymphocytes, B lymphocytes, plasma cells, and all their precursors.

Methods to introduce in vitro, ex vivo or in vivo proteins and nucleic acid molecules into cells are well known in the art. The traditional methods to introduce a nucleic acid or a protein in a cell include a vector such as a lentiviral vector or an AAV vector, or a protein microinjection, electroporation and sonoporation. Other techniques based on physical, mechanical and biochemical approaches such as magneto fection, optoinjection, optoporation, optical transfection and laserfection can also be mentioned (see Stewart M P et al, Nature, 2016).

Additionally, gene editing technologies such as zinc finger nucleases, meganucleases, TALENs, and CRISPR can also be used to integrate the nucleic acid molecule of the invention into the genome of the cell.

For example, a guide peptide-containing endonuclease may be introduced in the cell, such as transcription activator-like effector nuclease (TALEN) or a zinc-finger nuclease (ZFN). The TALENs technology comprises a non-specific DNA-cleaving domain (nuclease) fused to a specific DNA-binding domain The specific DNA-binding domain is composed of highly conserved repeats derived from transcription activator-like effectors (TALEs) which are proteins secreted by Xanthomonas bacteria to alter transcription of genes in host plant cells. The zinc-finger nuclease (ZFN) technology consists in the use of artificial restriction enzymes generated by fusion of a zinc finger DNA-binding domain to a DNA-cleavage domain (nuclease). The zinc finger domain specifically targets desired DNA sequences, which allows the associated nuclease to target a unique sequence within complex genomes.

In a preferred embodiment, the nucleic acid molecule of the invention is introduced into the cell, in combination with:
at least one single-stranded guide RNA binding to a selected target site and
a Clustered regularly interspaced short palindromic repeats (CRISPR) associated protein (Cas), in particular the CRISPR associated protein 9 (Cas9).

Mechanistically, the CRISPR/Cas9 system comprises two components, a single-stranded guide RNA (sgRNA) and a Cas9 endonuclease. The sgRNA often contains a unique 20 base-pair (bp) sequence designed to complement the target DNA site in a sequence-specific manner, and this must be followed by a short DNA sequence upstream essential for the compatibility with the Cas9 protein used, which is termed the “protospacer adjacent motif” (PAM) of an “NGG” or “NAG”.50,51 The sgRNA binds to the target sequence by Watson-Crick base pairing and Cas9 precisely cleaves the DNA to generate a double strand break before DNA-DSB repair mechanisms initiate genome repair.

### Pharmaceutical Composition

The present invention also provides pharmaceutical compositions comprising the nucleic acid molecule, the nucleic acid construct, the vector, the chimeric LAL polypeptide, or the cell of the invention.

Such compositions comprise a therapeutically effective amount of the therapeutic (the nucleic acid molecule, the nucleic acid construct, the vector, the chimeric LAL polypeptide or the cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject. In a particular embodiment, the nucleic acid, vector or cell of the invention is formulated in a composition comprising phosphate-buffered saline and supplemented with 0.25% human serum albumin In another particular embodiment, the nucleic acid, vector or cell of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection.

In an embodiment, the nucleic acid molecule, the nucleic acid construct, the vector, the chimeric LAL polypeptide or the cell of the invention can be delivered in a vesicle, in particular a liposome. In yet another embodiment, the nucleic acid molecule, the nucleic acid construct, the vector, the chimeric LAL polypeptide or the cell of the invention can be delivered in a controlled release system.

Methods of administration of the nucleic acid molecule, the nucleic acid construct, the vector, the chimeric LAL polypeptide or the cell of the invention include but are not limited to intradermal, intramuscular, intraperitoneal, intravenous, subcutaneous, intranasal, epidural, and oral routes. In a particular embodiment, the administration is via the intravenous or intramuscular route. The nucleic acid molecule, the nucleic acid construct, the vector, the chimeric LAL polypeptide or the cell of the invention, whether vectorized or not, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment, e.g. the liver. This may be achieved, for example, by means of an implant, said implant being of a porous, nonporous, or gelatinous material, including membranes, such as sialastic membranes, or fibers.

The amount of the therapeutic (i.e. the nucleic acid molecule, the nucleic acid construct, the vector, the chimeric LAL polypeptide or the cell of the invention) of the invention which will be effective in the treatment of a LAL-deficiency can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the nucleic acid molecule, the nucleic acid construct, the vector, the chimeric LAL polypeptide or the cell of the invention administered to the subject in need thereof will vary based on several factors including, without limitation, the route of administration, the specific disease treated, the subject's age or the level of expression necessary to obtain the therapeutic effect. One skilled in the art can readily determine, based on its knowledge in this field, the dosage range required based on these factors and others. In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×1012 vg/kg at least 1×1013 vg/kg, or at least 1×1014 vg/kg.

A further object of the present invention is a pharmaceutical composition comprising a genetically modified hematopoietic stem cell as described herein and/or at least one blood cell as described herein, in a pharmaceutically acceptable medium. In a particular embodiment, the cells as described herein can be administered in a composition as described herein with therapeutic compounds that augment the differentiation of the hematopoietic stem cell or progenitor cells. These therapeutic compounds have the effect of inducing differentiation and mobilization of hematopoietic stem cells and/or of progenitor cells that are endogenous, and/or the ones that are administered to the individual as part of the therapy.

Cells as described herein are administered into the subject by any suitable route, such as intravenous, intracardiac, intrathecal, intramuscular, intra-articular or intra-bone marrow injection, and in a sufficient amount to provide a therapeutic benefit. The amount of the cells needed for achieving a therapeutic effect will be determined empirically in accordance with conventional procedures for the particular purpose. Illustratively, administration of cells to a patient suffering from a LAL-deficiency provides a therapeutic benefit not only when the underlying condition is eradicated or ameliorated, but also when the patient reports a decrease in the severity or duration of the symptoms associated with the disease. Therapeutic benefit also includes halting or slowing the progression of the underlying disease or disorder, regardless of whether improvement is realized. The number of cells transfused will take into consideration factors such as sex, age, weight, the types of disease or disorder, stage of the disorder, the percentage of the desired cells in the cell population (e.g., purity of cell population), and the cell number needed to produce a therapeutic benefit. Generally, the numbers of cells infused may be from 1.10⁴ to 5.10⁶ cells/kg, in particular from 1.10⁵ to 10.10⁶ cells/kg, preferably from 5.10⁵ cells to about 5.10⁶ cells/kg of body weight.

### Treatment

The invention also relates to the nucleic acid molecule, the nucleic acid construct, the vector, the LAL chimeric polypeptide, the pharmaceutical composition or the cell of the invention, for use in a method for treating LAL-deficiency.

The invention further relates to a method for treating LAL-deficiency, which comprises a step of delivering a therapeutic effective amount of the nucleic acid molecule, the vector, the LAL chimeric polypeptide, the pharmaceutical composition or the cell of the invention to a subject in need thereof.

According to a particular embodiment, repeated administration of a therapeutic effective amount of the nucleic acid molecule, nucleic acid construct, vector, the LAL chimeric polypeptide, pharmaceutical composition or cell of the invention can be carried out. According to an embodiment, in the aspect comprising a repeated administration, said administration may be repeated at least once or more, and may even be considered to be done according to a periodic schedule, such as once per week, per month or per year. The periodic schedule may also comprise an administration once every 2, 3, 4, 5, 6, 7, 8, 9 or 10 year, or more than 10 years.

According to the present invention, a treatment may include curative, alleviation or prophylactic effects. Accordingly, therapeutic and prophylactic treatment includes amelioration of the symptoms of LAL-deficiency or preventing or otherwise reducing the risk of developing LAL-deficiency. The term “prophylactic” may be considered as reducing the severity or the onset of a particular condition. “Prophylactic” also includes preventing reoccurrence of a particular condition in a patient previously diagnosed with the condition. “Therapeutic” may also reduce the severity of an existing condition. The term ‘treatment’ is used herein to refer to any regimen that can benefit an animal, in particular a mammal, more particularly a human subject.

The invention also relates to the nucleic acid molecule, the nucleic acid construct, the vector, the LAL chimeric polypeptide, the pharmaceutical composition or the cell of the invention, for use in an ex vivo gene therapy method for the treatment of LAL-deficiency, comprising introducing the nucleic acid molecule or the nucleic acid construct of the invention into an isolated cell of a patient in need thereof, for example an isolated hematopoietic stem cell, and introducing said cell into said patient in need thereof. In a particular embodiment of this aspect, the nucleic acid molecule or construct is introduced into the cell with a vector as defined above. In a particular embodiment, the vector is an integrative viral vector. In a further particular embodiment, the viral vector is a retroviral vector, such as a lenviral vector. For example, a lentiviral vector as disclosed in van Til et al., 2010, Blood, 115(26), p. 5329, may be used in the practice in the method of the present invention.

The invention also relates to the nucleic acid molecule, the nucleic acid construct, the vector, the chimeric LAL polypeptide, the cell or the pharmaceutical composition of the invention for use as a medicament.

The invention also relates to the nucleic acid molecule, the nucleic acid construct, the vector, the chimeric LAL polypeptide, the cell or the pharmaceutical composition of the invention, for use in a method for treating a disease caused by a mutation in the LAL gene, in particular in a method for treating LAL-deficiency.

The invention further relates to the nucleic acid molecule, the nucleic acid construct, the vector, the chimeric LAL polypeptide, the cell or the pharmaceutical composition of the invention, for use in a method for treating early-onset or late-onset LAL-deficiency.

In a particular embodiment, the nucleic acid molecule, the nucleic acid construct, the vector, the chimeric LAL polypeptide, the cell or the pharmaceutical composition of the invention is for use in a method for treating Wolman Disease (WD).

In another particular embodiment, the nucleic acid molecule, the nucleic acid construct, the vector, the chimeric LAL polypeptide, the cell or the pharmaceutical composition of the invention is for use in a method for treating Cholesteryl Ester Storage Disease (CESD).

The chimeric LAL polypeptide of the invention may be administered to a patient in need thereof, for use in enzyme replacement therapy (ERT), such as for use in enzyme replacement therapy of LAL-deficiency, such as Wolman Disease (WD) or Cholesteryl ester storage disease (CESD).

In a preferred embodiment, the genetically modified HSC described above is administered to a patient in need thereof, for use in the treatment of a LAL-deficiency such as the Wolman Disease (WD) or the Cholesteryl ester storage disease (CESD). Thanks to this strategy, expression and secretion of therapeutic amounts of the LAL protein may be achieved in patients in need thereof.

The invention further relates to the use of the nucleic acid molecule, the nucleic acid construct, the vector, the chimeric LAL polypeptide or the cell of the invention, in the manufacture of a medicament useful for treating LAL-deficiency, such as for treating Wolman Disease (WD) or the Cholesteryl ester storage disease (CESD).

### EXAMPLES

The invention is further described in detail by reference to the following experimental examples and the attached figures. These examples are provided for purposes of illustration only, and are not intended to be limiting.

### Material and Methods

### Cell Culturing

K562 cells (ATCC® CCL-243) were maintained in RPMI 1640 medium containing 2 mM glutamine and supplemented with 10% fetal bovine serum (FBS, BioWhittaker, Lonza), 10 mM HEPES, 1 mM sodium pyruvate (LifeTechnologies) and penicillin and streptomycin (100 U/ml each, LifeTechnologies).

Mobilized peripheral blood- or cord blood-derived HSPC were thawed and cultured in prestimulation media for 48 h (StemSpan, Stemregenin-1 0.75 uM, StemCell technologies; rhSCF 300 ng/ml, Flt3-L 300 ng/ml, rhTPO 100 ng/ml and IL-3 20 ng/mL, CellGenix).

### Lentiviral Vector Description/Cloning

The expression cassette consisting of DNase I HSs HS2 (genomic coordinates[hg38], chr 11:5280255-5281665) and HS3 (genomic coordinates[hg38], chr11:5284251-5285452) of the βLCR and LIPA cDNA was clone into a pCCL LV backbone (Weber et al., Mol Ther Methods Clin Dev. 2018 Sep. 21; 10: 268-280.). Each cassette was synthetized by Genscript (Piscataway, NJ).

LV were produced by transient transfection of 293T using third-generation packaging plasmid pMDLg/p.RRE and pK.REV and pseudotyped with the vesicular stomatitis virus glycoprotein G (VSV-G) envelope. LV were titrated in HCT116 cells and HIV-1 Gag p24 content was measured by ELISA (Perkin-Elmer) according to manufacturer's instructions.

### Lentiviral Transduction

K562 cells were transduced overnight with lentiviral vector at a MOI 30 in presence of polybren, then cells were washed and disposed in a fresh medium.

HSPC were transduced overnight with lentiviral vectors at a MOI 75 in presence of rectronectin and protamilde sulfate, then cells were washed and cultured in erythroid differentiation medium (StemSpan, StemCell Technologies; SCF 20 ng/ml, Epo 1 u/mL, IL3 5 ng/ml, Dexamethasone 2 μM and Betaestradiol 1 μM; Sigma) for 14 days.

### Western Blot

To detect intracellular proteins cells were lysed in RIPA buffer (Sigma Aldrich) supplemented with protease inhibitor (Roche), freezed/thawed and centrifuged 10′ at 14 000 at 4° C. Total protein was quantified using BCA assay (Thermofisher). 5-15 μg of protein or 2.5 ul of supernatant were denatured at 90° C. for 10′, run under reducing conditions on a 4-12% Bis-tris gel and transferred to a nitrocellulose membrane using iBlot2 system (Invitrogen). After Ponceau staining (Invitrogen) membranes were blocked for 2 hours with Odyssey blocking buffer (Odyssey Blocking buffer (PBS), Li-Cor Biosciences) and incubated for 1 hour with primary antibodies against human LAL followed by specific secondary antibodies in PBS:Blocking buffer (see table 1 below). β-Tubulin was used as loading control. Blots were imaged at 169 μm with Odyssey imager and analyzed with ImageStudio Lite software (Li-Cor Biosciences). After image background subtraction (average method, top/bottom), band intensities were quantified and normalized with tubulin signal.

TABLE 1

| | | Fluoro- | | |
|---|---|---|---|---|
| Target | Host | chrome | Clone | Company |
| LAL | Mouse | — | 9G7F12, 7G6D7 | Thermofisher |
| β-tubulin | Rabbit | — | Polyclonal | Li-Cor |
| | | | | Biosciences |
| Anti-mouse | Goat | 800CW | Polyclonal | Li-Cor |
| IgG (H + L) | | | | Biosciences |
| Anti-rabbit | Goat | 680LT | Polyclonal | Li-Cor |
| IgG (H + L) | | | | Biosciences |

### LAL Activity

Samples were incubated 10 min at 37° C. with 42 μM Lalistat-2 (Sigma Aldrich), a specific competitive inhibitor of LAL, or water. Samples were then transferred to a Optiplate 96F plate (PerkinElmer) where fluorimetric reactions were initiated with 75 ul of substrate buffer (340 μM 4-MUP, 0.9% Triton X-100 and 12.9 μM cardiolipin in 135 mM acetate buffer pH 4.0). After 10 minutes, fluorescence was recorded (35 cycles, 30″ intervals, 37° C.) using SPARK TECAN Reader (Tecan, Austria). Kinetic parameters (average rate) were calculated using Magellan Software. LAL activity over untreated samples was quantified using this formula:

$$\\ \frac{Edited sample ( without Lalistat - With Lalistat )}{Untreated sample ( without Lalistat - With Lalistat )}$$

Co-Culture of Transduced K562 with Patient Fibroblasts

Transduced K562 cells were applied in a 0.4 μm insert disposed on the top of patient fibroblasts (Wolman patient; GM 11851A; health donor: GM 08333C, Coriell institute) cultured for 24 h in 24-well plate. K562 and patient fibroblasts were co-cultured for 3 days in opti-MEM medium (Gibco), then the inserts were removed and fibroblast cells were stained with Nile red. 8 fields for each condition were randomly acquired with an inverted fluorescence microscope (EVOS, 10×0 magnification) and average fluorescence intensity per cell was calculated with a custom-made ImageJ plugin.

### Chimeric LAL Proteins

The expression, secretion and activity of several chimeric LAL proteins were tested. The endogenous signal peptide sequence of the LAL protein was replaced with several different signal peptide sequences coming from different human proteins (Table 2).

TABLE 2

| Peptide | | | |
|---|---|---|---|
| signal | | | |
| (SP) | Name | Sequence | SEQ ID NO |
| Sp 1 | LAL WT | MKMRFLGLVVCLVLWTLHSEGSG | SEQ ID NO: 1 |
| Sp 2 | alpha-1-antytripsin | MPSSVSWGILLLAGLCCLVPVSLA | SEQ ID NO: 2 |
| Sp 6 | iduronate 2-sulfatase | MPPPRTGRGLLWLGLVLSSVCVALG | SEQ ID NO: 3 |
| Sp 7 | chymotrypsinogen B2 | MAFLWLLSCWALLGTTFG | SEQ ID NO: 4 |
| Sp 8 | plasma protease | MASRLTLLTLLLLLLAGDRASS | SEQ ID NO: 5 |
| | inhibitor | | |
| Sp 9 | serum albumin | MKWVTFISLLFLFSSAYS | SEQ ID NO: 6 |
| | preproprotein | | |
| Sp 10 | azurocidin | MTRLTVLALLAGLLASSRA | SEQ ID NO: 7 |
| | preproprotein | | |

In the figures, “Sp 1”, “Sp 2”, “Sp 6”, “Sp 7”, “Sp8”, “Sp9” and “Sp 10” refer to a chimeric LAL protein comprising a functional LAL moiety peptide of SEQ ID NO:9 fused to the corresponding signal peptide as referred in Table 2.

Several codon-optimized sequences encoding for a chimeric LAL protein comprising Sp 8 as a signal peptide were also tested. In the figures, “Sp8_Opt 1”, “Sp8_Opt2” and “Sp8_Opt3” refer to codon optimized sequences of SEQ ID NO:27, SEQ ID NO:28 and SEQ ID NO:29 respectively. SEQ ID NO:27 comprises a KOZAK sequence (CACC), the optimized sequence of SEQ ID NO:21 and an HA-tag coding sequence. SEQ ID NO:28 comprises a KOZAK sequence (CACC), the optimized sequence of SEQ ID NO:22 and an HA-tag coding sequence. SEQ ID NO:29 comprises a KOZAK sequence (CACC), the optimized sequence of SEQ ID NO:23 and an HA-tag coding sequence.

Expression, secretion and activity of the protein encoded by these optimized sequences were evaluated, and normalized on the expression, secretion and activity of a proteins expressed from a non-optimized sequence of SEQ ID NO:33 encoding a chimeric LAL protein comprising Sp8.

### Results and Discussion

In order to ameliorate the secretion of the human LAL enzyme, we replaced, in the human LAL cDNA sequence, the endogenous signal peptide with signal peptides coming from different secreted human proteins. These cDNA were inserted in a lentiviral vector under the control of the artificial human beta globin promoter (Miccio A. et al, PNAS 2008) for erythroid expression. K562 human erythroleukemia cell line was transduced with a similar amount of the different lentiviral vector and, after 6 days, cells were lysed to extract protein for quantification (western blot) and enzymatic activity (using fluorogenic substrate).

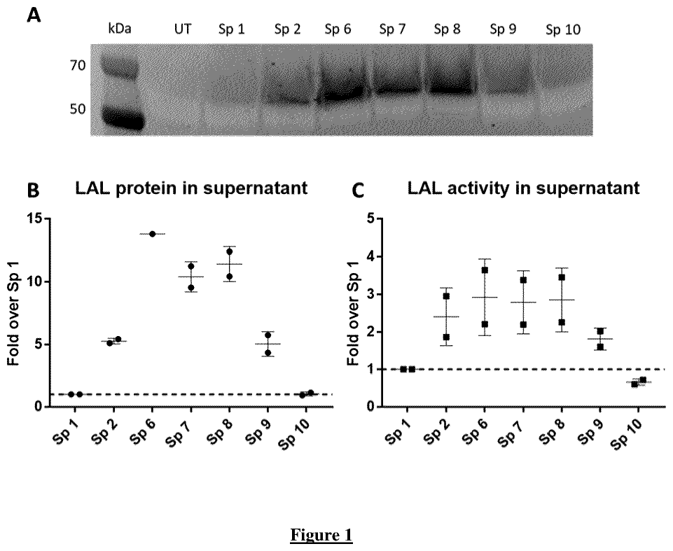

In FIG. 1 we can observe that heterologous signal peptides sp6, sp7 and sp8 increase both protein expression and protein secretion (protein in supernatant). Enzymatic activity in supernatant is also importantly increased with the heterologous signal peptides sp6, sp7 and sp8. On the contrary, heterologous signal peptides sp2, sp9 and sp10 does not allow such improvements. Sp10 even leads to a decrease of the enzymatic activity as compared to the wild type sequence.

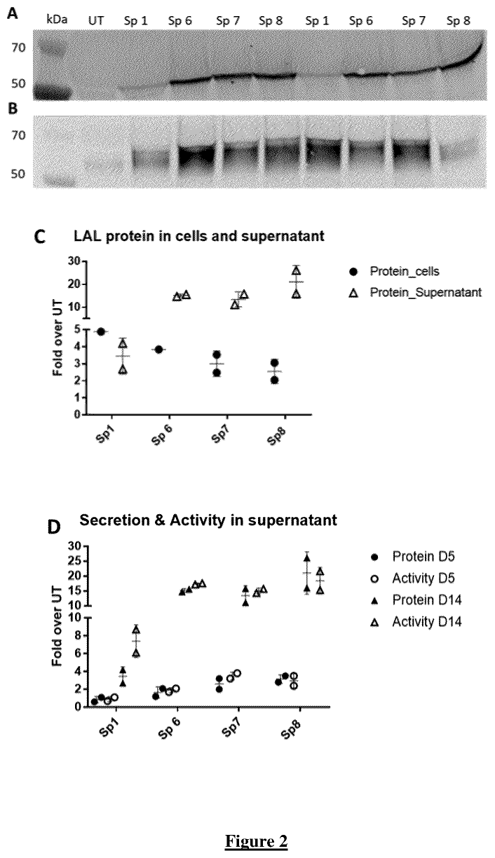

To confirm this result in clinically relevant cells, we transduced primary human hematopoietic stem/progenitor cells with selected lentiviral vectors. Again, we observed a clear increase in protein expression, secretion and activity in cells supernatant when using heterologous signal peptides sp6, sp7 and sp8 (FIG. 2). We can see that, signal peptides sp6, sp7 and sp8 led to an impressive increase of secreted LAL protein . In addition, sp6, sp7 and sp8 allow not only a better LAL secretion but also a better global expression of LAL (intracellular LAL+secreted LAL), when compared to sp 1 signal peptide. This dual effect obtained with sp6, sp7 and sp8signal peptides is unexpected for the skilled person.

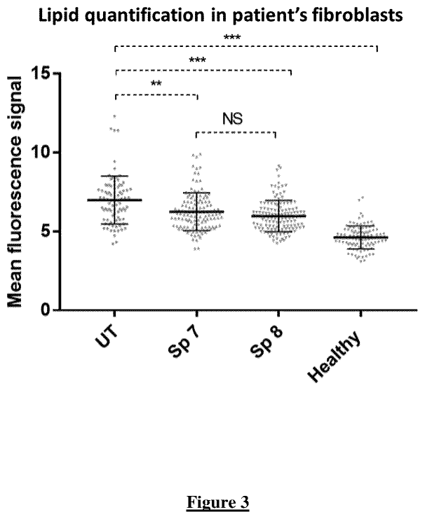

To confirm the full functionality of these chimeric LAL proteins, we confirmed cross-correction ability by exposing patient's primary fibroblasts to the chimeric enzymes secreted from transduced K562 cells FIG. 3 shows that chimeric LAL proteins comprising sp7 and sp8 allow restoring lipid degradation, compared to untreated cells, thus confirming functionality of chimeric LAL proteins.

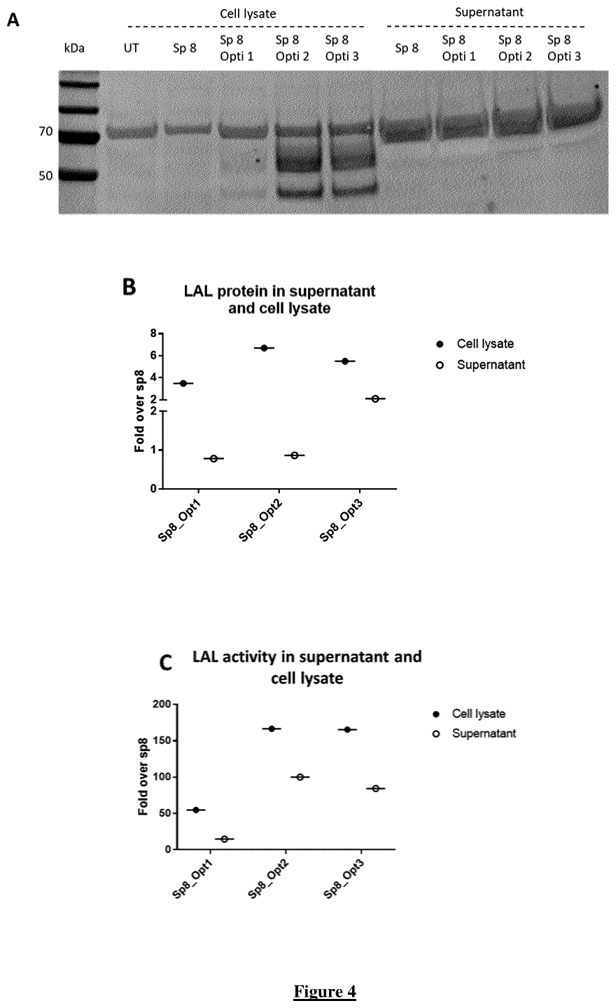

Lastly, to further increase LAL expression and secretion, we generated 3 codon optimized versions of LAL cDNA and we confirmed that at least two of them led to a significant increase in enzyme expression (FIG. 4).

Overall, we improved hLAL secretion and expression by engineering its nucleotide sequence and signal peptide without affecting enzyme functionality.

### Conclusion

In this study, the LIPA gene was modified with the aim to improve its secretion.

In particular, the endogenous signal peptide sequence of the LAL protein was replaced with several different sequences derived from different human proteins (Table 2) and the effect on enzyme secretion on K562 human erythroleukemia cell line (FIG. 1) and primary hematopoietic stem/progenitor cells (HSPC, FIG. 2) was tested. It is herein shown that signal peptides SP6, SP7 and SP8 significantly increase the secretion of the protein without affecting its enzymatic activity.

Moreover, the results show that said chimeric enzymes were still capable of cross-correction. In fact, co-culturing fibroblasts derived from Wolman patient with K562 cells expressing the chimeric enzymes resulted in a reduction of pathological lipid accumulation.

Overall, these data indicate that these chimeric enzymes not only are better expressed and secreted, but they remain functional and can be uptaken by affected cells and functionally correct them.

Finally, the possibility of increasing enzyme expression by codon optimization of the LIPA sequence was evaluated. 3 novel transgenes were designed (Opt_1, Opt_2 and Opt_3) and at least 2 performed better than the wild type sequence in K562 (FIG. 4).

These two parameters could allow for a larger fraction of functional enzyme in the bloodstream, which should lead to a greater therapeutic effect. Said results open the path for an effective long-term curative strategy for LAL deficiency. In particular, said chimeric LAL peptides may be used in combination with gene therapy vectors allowing in vivo expression and replacement of the deficient enzyme. Said chimeric LAL peptides may also be used for ex vivo gene therapy aiming at introducing a functional copy of the modified LIPA gene into patients' hematopoietic stem cells (HSCs). The corrected HSCs will be administered into the bloodstream, reach and multiply in the bone marrow to produce new corrected cells in the blood. As a result, the chimeric LAL enzyme will be efficiently expressed and secreted into the bloodstream in order to be uptaken by LAL-deficient cells to restore metabolic function.

1-18. (canceled).

19. A nucleic acid molecule encoding a functional chimeric LAL protein, comprising a signal peptide moiety and a functional LAL moiety, wherein the signal peptide moiety has an amino acid sequence selected from the group consisting of SEQ ID NOs:3, 4 and 5, or wherein the signal peptide moiety is a functional signal peptide moiety having an amino acid sequence comprising from 1 to 5 amino acid deletion(s), insertion(s) or substitution(s) as compared to the sequence of SEQ ID NO:3, 4 or 5.

20. The nucleic acid molecule of claim 19, wherein the functional LAL moiety is a functional human LAL moiety, optionally devoid of its natural signal peptide.

21. The nucleic acid molecule of claim 19, wherein the functional LAL moiety comprises SEQ ID NO:9 or has at least 75% sequence identity to the sequence as shown in SEQ ID NO:9.

22. The nucleic acid molecule of claim 19, comprising a nucleotide sequence resulting from the combination of:
a signal peptide moiety coding sequence selected from the group consisting of SEQ ID NOs:11, 12, 13, 18, 19 and 20; and
a LAL moiety coding sequence selected from the group consisting of SEQ ID NOs:14, 15, 16, and 17.

23. The nucleic acid molecule of claim 19, which is a nucleotide sequence optimized to improve the expression of the functional chimeric LAL protein in vivo.

24. The nucleic acid molecule of claim 23, wherein the nucleotide sequence comprises SEQ ID NO:21, SEQ ID NO:22 or SEQ ID NO:23.

25. A nucleic acid construct comprising the nucleic acid molecule of claim 19, said construct comprising an expression cassette comprising said nucleic acid molecule operably linked to a promoter, wherein said nucleic acid construct optionally further comprises an intron and/or post-transcriptional regulatory sequence(s).

26. A vector comprising the nucleic acid molecule of claim 19 or a nucleic acid construct comprising said nucleic acid molecule, wherein the vector is a viral vector.

27. The vector of claim 26, wherein the viral vector is a retroviral vector, a lentiviral vector, an adenoviral vector, an AAV vector or a single-stranded or double-stranded self-complementary AAV vector.

28. The vector of claim 27, wherein the single-stranded or double-stranded self-complementary AAV vector is an AAV vector with an AAV-derived capsid or an AAV6, AAV8, AAV9, AAV2 or AAV-DJ derived capsid.

29. A cell comprising the nucleic acid molecule of claim 19; a nucleic acid construct comprising said nucleic acid molecule; or a vector comprising said nucleic acid molecule or said nucleic acid construct.

30. The cell of claim 29, which is a genetically modified hematopoietic stem cell comprising the nucleic acid molecule in at least one globin locus, wherein said nucleic acid molecule is placed under the control of the endogenous promoter of a globin gene.

31. A functional chimeric LAL protein, comprising a signal peptide moiety and a functional LAL moiety, wherein the signal peptide moiety has an amino acid sequence selected from the group consisting of SEQ ID NOs:3, 4, and 5, or wherein the signal peptide moiety is a functional signal peptide moiety having an amino acid sequence comprising from 1 to 5 amino acid deletion(s), insertion(s) or substitution(s) as compared to the sequence of SEQ ID NO:3, 4 or 5.

32. The functional chimeric LAL protein of claim 31, wherein the functional LAL moiety is a functional human LAL moiety, optionally devoid of its natural signal peptide or comprising SEQ ID NO:9, or having at least 75% sequence identity to SEQ ID NO:9.

33. The functional chimeric LAL protein of claim 31, said functional chimeric LAL protein comprising an amino acid sequence resulting from the combination of:
a signal peptide moiety sequence selected from the group consisting of SEQ ID NOs:3, 4 and 5; and
a LAL moiety sequence of SEQ ID NO:9.

34. A pharmaceutical composition, comprising, in a pharmaceutically acceptable carrier, the nucleic acid molecule of claim 19, a nucleic acid construct comprising said nucleic acid molecule, a vector comprising said nucleic acid molecule or said nucleic acid construct or a cell comprising said nucleic acid, a nucleic acid construct comprising said nucleic acid or a vector comprising said nucleic acid or nucleic acid construct.

35. A pharmaceutical composition comprising, in a pharmaceutically acceptable carrier, the functional chimeric LAL protein of claim 31.

36. A method of treating a LAL-deficiency comprising administering the nucleic acid molecule of claim 19, a nucleic acid construct comprising said nucleic acid molecule, a vector comprising said nucleic acid molecule or said nucleic acid construct or a cell comprising said nucleic acid, a nucleic acid construct comprising said nucleic acid or a vector comprising said nucleic acid or nucleic acid construct to a subject in need of treatment.

37. The method of claim 36, wherein the LAL-deficiency is Wolman Disease (WD) or Cholesteryl Ester Storage Disease (CESD).

38. The method of claim 36, wherein the cell is an autologous hematopoietic stem cell.

39. A method for the ex vivo preparation of a genetically modified hematopoietic stem cell comprising transforming a hematopoietic stem cell with a nucleic acid molecule of claim 19, a nucleic acid construct comprising said nucleic acid molecule, or a vector comprising said nucleic acid molecule or said nucleic acid construct.

40. A method of treating a LAL-deficiency comprising administering functional chimeric LAL protein of claim 31 or a pharmaceutical composition comprising functional LAL protein to a subject in need of treatment.
